(19) United States
(12) Patent Application Publication
(10) Pub. No.: US 2012/0288482 A1
(43) Pub. Date: Nov. 15, 2012

(54) THERAPEUTIC AGENT (Y-39983) FOR CORNEAL ENDOTHELIAL DYSFUNCTION

(75) Inventors: Hiroaki Takahashi, Hyogo (JP); Yuji Sakamoto, Hyogo (JP); Tetsuo Kida, Hyogo (JP); Takeshi Tarui, Hyogo (JP)

(73) Assignees: MITSUBISHI TANABE PHARMA CORPORATION, Osaka-shi, Osaka (JP); SENJU PHARMACEUTICAL CO., LTD., Osaka-shi, Osaka (JP)

(21) Appl. No.: 13/519,682

(22) PCT Filed: Dec. 28, 2010
(86) PCT No.: PCT/JP2010/073904

§ 371 Date: Aug. 6, 2012

(30) Foreign Application Priority Data
Dec. 29, 2009 (JP) 2009-299180
Nov. 24, 2010 (JP) PCT/JP2010/071424

(51) Int. Cl. A61K 31/437; A61K 35/44; A61P 27/02

(52) U.S. Cl. 424/93.7; 514/300

(57) ABSTRACT

The present invention aims to provide a means for effectively and conveniently treating diseases wherein corneal endothelial cells poor in proliferative capacity in vivo are damaged. The present invention provides a therapeutic agent for corneal endothelial dysfunction containing (R)-(+)-N-(1H-pyrrolo [2,3-b]pyridin-4-yl)-4-(1-aminoethyl)benzamide <Y-39983> or a pharmacologically acceptable salt thereof (compound (Ia)) as active ingredient, an agent for promoting adhesion of corneal endothelial cells, containing compound (Ia), a culture medium for corneal endothelial cells, containing the agent for promoting adhesion, an implant for corneal endothelial keratoplasty, containing corneal endothelial cells, scaffold and compound (Ia), and a production method of a corneal endothelial preparation, including a step of cultivating corneal endothelial cells using the culture medium.

### TECHNICAL FIELD

The present invention relates to a therapeutic agent for corneal endothelial dysfunction. In particular, the therapeutic agent for corneal endothelial dysfunction of the present invention is used for healing wound of the corneal endothelium or adhesion, maintenance or preservation of corneal endothelial cells.

### BACKGROUND ART

Visual information is recognized when the light that entered from the cornea (transparent tissue at the forefront of the eyeball) reaches the retina to excite retinal neuronal cells, and the developed electric signals are transmitted to the cerebral visual cortex via the optic nerve. To have good visual acuity, the cornea needs to be transparent. The transparency of the cornea is kept by maintaining the homeostasis of a 3-layer structure of corneal epithelium, stroma and endothelium. Of these, corneal endothelial cells keep water content of the cornea at a constant level, and are important cells that maintain transparency of the cornea. However, human corneal endothelial cells are poor in the proliferative capacity in vivo, and suffer from irreversible corneal endothelium functional disorders due to diseases, trauma and injury by ophthalmic surgery.

It has been reported that, in cultured corneal endothelial cells, Y-27632, which is a selective Rho kinase (ROCK) inhibitor, has an effect of promoting cell adhesion (non-patent document 1). In addition, it has been reported that instillation of 10 mM Y-27632 promotes wound healing of corneal endothelium in rabbit corneal endothelial wound healing model (non-patent document 2).

As for Y-27632 and Fasudil, which are Rho kinase inhibitors, actions in vitro have been reported, such as

1) cultivation of corneal endothelial cells (rabbit, monkey etc.),
2) promotion of cell adhesion in cultured monkey corneal endothelial cells,
3) promotion of cell cycle in cultured monkey corneal endothelial cells,
4) suppression of apoptosis in cultured monkey corneal endothelial cells, and
5) possible application of cultured corneal endothelial cells to the treatment of diseases requiring corneal endothelial keratoplasty (patent document 1).
Patent document 1 does not describe the in vivo action of Y-27632 and Fasudil. In addition, an influence of Rho kinase inhibitors other than Y-27632 and Fasudil on the corneal endothelial cells is not considered.

patent document 1: WO2009/028631

non-patent document 1: Okumura N, et al., Invest Ophthalmol Vis Sci. 2009, 50(8) p.3680-7

non-patent document 2: Invest Ophthalmol Vis Sci. 2009, 50: E-Abstract 1817.

### SUMMARY OF INVENTION

### Technical Problem

An object of the present invention is to provide a means for effectively and conveniently treating diseases wherein corneal endothelial cells poor in proliferative capacity in vivo are damaged.

### Solution to Problem

The present inventors have conducted intensive studies in view of the above-mentioned problems and found that a particular compound in Rho kinase inhibitors can cure corneal endothelial wound at a small dose or low concentration. In addition, the present inventors have found that the compound can exhibit a sufficient wound healing effect even when administered at a remarkably lower concentration than that of conventional Rho kinase inhibitors to the body by topical instillation through corneal epithelium, and succeeded in utilizing the compound for an implant for corneal endothelial keratoplasty, a corneal endothelial preparation and the like, which resulted in the completion of the present invention. Accordingly, the present invention is as follows.

[1] A therapeutic agent for a corneal endothelial dysfunction, comprising a compound represented by the following formula (1):

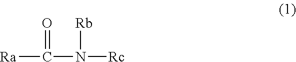

wherein Ra is the formula (2):

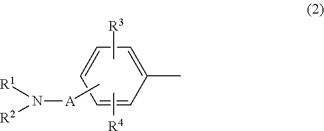

wherein R¹ is a hydrogen, an alkyl, or a cycloalkyl, a cycloalkylalkyl, a phenyl or an aralkyl, which optionally has a substituent on a ring, or a group of the formula (3):

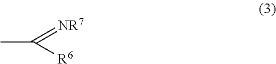

wherein R⁶ is hydrogen, alkyl or the formula: —NR⁸R⁹ wherein R⁸ and R⁹ are the same or different and each is hydrogen, alkyl, aralkyl or phenyl, and R⁷ is hydrogen, alkyl, aralkyl, phenyl, nitro or cyano, or R⁶ and R⁷ combinedly form a heterocycle optionally having oxygen atom, sulfur atom or optionally substituted nitrogen atom additionally in the ring; R² is a hydrogen, an alkyl, or a cycloalkyl, a cycloalkylalkyl, a phenyl or an aralkyl, which optionally has a substituent on the ring; or
R¹ and R² combinedly form, together with the adjacent nitrogen atom, a heterocycle optionally having oxygen atom, sulfur atom or optionally substituted nitrogen atom additionally in the ring;
R³ and R⁴ are the same or different and each is a hydrogen, an alkyl, an aralkyl, a halogen, a nitro, an amino, an alkylamino, an acylamino, a hydroxy, an alkoxy, an aralkyloxy, a cyano, an acyl, a mercapto, an alkylthio, an aralkylthio, a carboxy, an alkoxycarbonyl, a carbamoyl, an alkylcarbamoyl or an azide;
A is the formula (4):

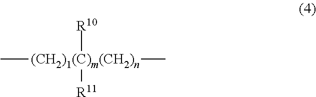

wherein R¹⁰ and R¹¹ are the same or different and each is hydrogen, alkyl, haloalkyl, aralkyl, hydroxyalkyl, carboxy or alkoxycarbonyl, or
R¹⁰ and R¹¹ combinedly form cycloalkyl, and l, m and n are each 0 or an integer of 1-3;
Rb is a hydrogen or an alkyl; and
Rc is an optionally substituted heterocycle containing nitrogen,
or a pharmacologically acceptable salt thereof (hereinafter to be referred to as compound (1)) as an active ingredient.
[2] The therapeutic agent of the above-mentioned [1], wherein the above-mentioned corneal endothelial dysfunction is bullous keratopathy or corneal endotheliitis.
[3] The therapeutic agent of the above-mentioned [1] or [2], which is an eye drop.
[4] The therapeutic agent of any of the above-mentioned [1] to [3], wherein the above-mentioned compound (1) is (R)-(+)-N-(1H-pyrrolo[2,3-b]pyridin-4-yl)-4-(1-aminoethyl)benzamide (hereinafter to be referred to as compound (Ia)).
[5] An agent for promoting adhesion of corneal endothelial cells, comprising compound (1).
[6] The agent of the above-mentioned [5], wherein the above-mentioned compound (1) is compound (Ia).
[7] A culture medium for corneal endothelial cells, comprising compound (1).

[8] The culture medium of the above-mentioned [7], wherein the above-mentioned compound (1) is compound (Ia).

[9] A corneal storage solution comprising compound (1).
[10] The corneal storage solution of the above-mentioned [9], wherein the above-mentioned compound (1) is compound (Ia).
[11] An implant for corneal endothelial keratoplasty, comprising
A) corneal endothelial cells,
B) scaffold, and
C) compound (1).
[12] The implant of the above-mentioned [11], wherein the above-mentioned corneal endothelial cells are derived from human.
[13] The implant of the above-mentioned [11] or [12], wherein the above-mentioned compound (1) is compound (Ia).
[14] A method of producing a corneal endothelial preparation, comprising a step of cultivating corneal endothelial cells using a culture medium containing compound (1).
[15] The production method of the above-mentioned [14], wherein the above-mentioned corneal endothelial cells are derived from human.
[16] The production method of the above-mentioned [14] or [15], wherein the above-mentioned compound (1) is compound (Ia).
[17] A method of treating a corneal endothelial dysfunction, comprising a step of providing a corneal endothelial preparation and/or an implant for corneal endothelial keratoplasty each comprising compound (1), and a step of transplanting the preparation and/or the implant into a subject in need of the keratoplasty.
[18] The treatment method of the above-mentioned [17], wherein the above-mentioned corneal endothelial cells are derived from human.
[19] The treatment method of the above-mentioned [17] or [18], wherein the above-mentioned corneal endothelial dysfunction is bullous keratopathy, corneal edema or corneal leukoma.
[20] The treatment method of any of the above-mentioned [17] to [19], wherein the above-mentioned compound (1) is compound (Ia).
[21] A method of treating a corneal endothelial dysfunction, comprising a step of administering an effective amount compound (1) and corneal endothelial cells to a subject in need of corneal endothelial wound healing.
[22] The treatment method of the above-mentioned [21], wherein the above-mentioned corneal endothelial dysfunction is bullous keratopathy or corneal endotheliitis.
[23] The treatment method of the above-mentioned [21] or [22], wherein the above-mentioned administration step is topical instillation.
[24] The treatment method of any of the above-mentioned [21] to [23], wherein the above-mentioned compound (1) is compound (Ia).
[25] Use of compound (1) for producing a therapeutic agent for a corneal endothelial dysfunction.
[26] The use of the above-mentioned [25], wherein the above-mentioned therapeutic agent is an eye drop.
[27] Use of compound (1) for producing an agent for promoting adhesion of corneal endothelial cells.
[28] Use of compound (1) for producing a culture medium for corneal endothelial cells.

### [29] Use of

A) corneal endothelial cells,
B) scaffold, and
C) compound (1)
for producing an implant for corneal endothelial keratoplasty.
[30] The use of the above-mentioned [29], wherein the above-mentioned corneal endothelial cells are derived from human.
[31] The use of any of the above-mentioned [25] to [30], wherein the above-mentioned compound (1) is compound (Ia).
[32] A corneal endothelial preparation obtained by the production method of any of the above-mentioned [14] to [16].
[33] An intraocular irrigating solution comprising compound (1).
[34] The intraocular irrigating solution of the above-mentioned [33], wherein the above-mentioned compound (1) is compound (Ia).
[35] An apoptosis suppressor comprising compound (1).
[36] The apoptosis suppressor of the above-mentioned [35], wherein the above-mentioned compound (1) is compound (Ia).
[37] A kit for the treatment of a corneal endothelial dysfunction, comprising compound (1), corneal endothelial cells and an instruction.
[38] The kit of the above-mentioned [37], wherein the above-mentioned corneal endothelial cells are frozen.
[39] The kit of the above-mentioned [37] or [38], wherein the above-mentioned compound (1) is contained in a washing solution, a culture medium or a solution for cell suspension.
[40] The kit of any of the above-mentioned [37] to [39], wherein compound (1) is compound (Ia).
[41] A corneal endothelial preparation comprising compound (1) and corneal endothelial cells.
[42] The corneal endothelial preparation of the above-mentioned [41], wherein the above-mentioned compound (1) is compound (Ia).
[43] Compound (1) for the treatment of a corneal endothelial dysfunction.
[44] Compound (Ia) for the treatment of a corneal endothelial dysfunction.

### ADVANTAGEOUS EFFECTS OF INVENTION

The therapeutic agent for a corneal endothelial dysfunction of the present invention comprises compound (1), preferably compound (Ia), as an active ingredient. As a result, an effective and convenient method for the treatment or prophylaxis of a disease with disordered corneal endothelial cells, that is, a disease associated with corneal endothelial dysfunction (e.g., bullous keratopathy, corneal endotheliitis etc.) can be provided. Compound (Ia) to be contained in the therapeutic agent for the corneal endothelial dysfunction of the present invention can exhibit efficacy even at a low concentration of about 1/30- 1/10 of Y-27632 in the case of topical instillation. Thus, even a topical instillation type dosage form is expected to reach corneal endothelium through corneal epithelium and retain its action. Using the therapeutic agent for a corneal endothelial dysfunction of the present invention can provide increasing options of the administration route and a superior treatment as long-acting substance.

The agent for promoting adhesion of corneal endothelial cells of the present invention is useful as an agent for protecting corneal endothelium in the prophylaxis or treatment of a disease accompanied by a corneal endothelial dysfunction. Furthermore, the agent for promoting adhesion of corneal endothelial cells of the present invention can be utilized as an agent for protecting corneal endothelium in the prophylaxis or treatment of corneal endothelial dysfunction associated with an intraocular surgery such as cataract surgery, vitreous surgery and the like, corneal endothelial dysfunction caused by increased intraocular pressure (particularly glaucomatous attack), or corneal endothelial dysfunction caused by insufficient oxygen due to contact lenses. Since the culture medium of the present invention contains a compound (1), preferably compound (Ia), corneal endothelial cells can be cultured, maintained or preserved fine, and stable supply, maintenance or preservation of a corneal endothelial preparation is enabled.

The implant for corneal endothelial keratoplasty of the present invention can produce the form of, for example, a corneal endothelium sheet in vitro, and can be provided for corneal endothelial keratoplasty together with corneal endothelial cells and scaffold thereof, as an implant for corneal endothelial keratoplasty. The implant for corneal endothelial keratoplasty of the present invention has the characteristics of the intravital corneal endothelial cell layer, and is expected to improve the engrafted rate of an implant.

### BRIEF DESCRIPTION OF DRAWINGS

FIG. 1 shows alizarin red-stained images showing the effect of various compounds on the corneal endothelial wound of rabbit corneal endothelial wound model. Topical instillation of negative control (PBS): FIG. 1A, 10 mM Y-27632: FIG. 1B, 10 mM Fasudil: FIG. 1C, 0.32 mM compound (I): FIG. 1D, and 0.95 mM compound (I): FIG. 1E.

FIG. 2 is a graph showing the topical instillation effect of compound (I) in rabbit corneal endothelial wound model, wherein the vertical axis shows corneal endothelium defective area.

FIG. 3 shows an influence of compound (I) on the morphology of cultured corneal endothelial cells (one day after seeding).

FIG. 4 shows an influence of compound (I) on the morphology of cultured corneal endothelial cells (3 days after seeding).

FIG. 5 shows an influence of compound (I) on the morphology of cultured corneal endothelial cells (5 days after seeding).

FIG. 6 shows an influence of compound (I) on the morphology of cultured corneal endothelial cells (7 days after seeding).

FIG. 7 shows an influence of compound (I) on the morphology of cultured corneal endothelial cells (14 days after seeding).

FIG. 8 shows changes in the wound width of corneal endothelial cells after addition of medicament, wherein the vertical axis shows the ratio (%) of wound width after the addition of medicament to that before the addition, and the horizontal axis shows the medicament added. In each group, the ratios of the wound width at 0 hr (before addition), 6 hrs (6 hours after addition), 12 hrs (12 hours after addition) and 24 hrs (24 hours after addition) are shown from the left.

FIG. 9 shows the numbers of corneal endothelial cells adhered to the well for 3 hours after the seeding, wherein the vertical axis shows the rate (%) of cell count relative to the cell count of the control group as 100, and the horizontal axis shows the medicament added.

FIG. 10 shows immunostained images of ZO-1 and Na⁺/K⁺ ATPase in culture corneal endothelial cell sheet for transplantation prepared after 48 hours by adding compound (I), Y-27632 and DMSO, wherein FIG. 10-(A) shows ZO-1 staining on addition of various medicaments, and FIG. 10-(B) shows Na⁺/K⁺ ATPase staining.

FIG. 11 shows immunostained images of ZO-1 and Na⁺/K⁺ATPase in culture corneal endothelial cell sheets for transplantation prepared after 14 days by adding compound (I) and DMSO.

FIG. 12 shows immunostained images of corneal endothelium after 14 days from injection of corneal endothelial cells into rabbit bullous keratopathy model, wherein FIG. 12-(A) shows Phalloidin staining, and FIG. 12-(B) shows Na⁺/K⁺ ATPase staining.

FIG. 13 shows corneal endothelial cell count after 14 days from injection of corneal endothelial cells into rabbit bullous keratopathy model, wherein the vertical axis shows cell count (cells/mm²), and the bars in the graph show control group, 100 μM Y-27632 treatment group and 10 μM compound (I) treatment group from the left.

FIG. 14 shows changes in the wound width of corneal endothelial cells after addition of medicament, wherein the vertical axis shows the ratio (%) of wound width after the addition of medicament to that before the addition, and the horizontal axis shows the medicament added. In each group, the ratios of the wound width at 0 hr (before addition), 6 hrs (6 hours after addition), 12 hrs (12 hours after addition) and 24 hrs (24 hours after addition) are shown from the left.

FIG. 15 shows the stained images with Hoechst, PI and Annexin V of the cornea preserved for 3 weeks in a storage solution added with compound (I) or Y-27632.

FIG. 16 shows the numbers of living cells, dead cells and apoptotic cells in the cornea preserved for 2 weeks in a storage solution added with compound (I) or Y-27632. The left graph shows the results obtained using the storage solution added with compound (I), and the right graph shows the results obtained using the storage solution added with Y-27632. In each graph, the vertical axis shows the cell count and the horizontal axis shows the staining agents used for identification of cells.

FIG. 17 shows the numbers of living cells, dead cells and apoptotic cells in the cornea preserved for 3 weeks in a storage solution added with compound (I) or Y-27632. The left graph shows the results obtained using the storage solution added with compound (I), and the right graph shows the results obtained using the storage solution added with Y-27632. In each graph, the vertical axis shows the cell count and the horizontal axis shows the staining agents used for identification of cells.

### DESCRIPTION OF EMBODIMENTS

The present invention is described below. It should be understood that, throughout the present specification, the articles for singular forms include the concept of their plurality unless otherwise specified. Therefore, articles for singular forms (e.g., “a”, “an”, “the”, and the like in English) include the concept of their plurality unless otherwise specified. It should also be understood that terms as used herein have definitions ordinarily used in the art unless otherwise specified. Therefore, all technical and scientific terms used herein have the same meanings as commonly understood by those skilled in the relevant art. Otherwise, the present specification (including definitions) takes precedence.

In one aspect, the present invention provides a therapeutic agent for corneal endothelial dysfunction. The therapeutic agent for corneal endothelial dysfunction of the present invention (hereinafter to be sometimes referred to as “the therapeutic agent of the present invention”) contains compound (1) as an active ingredient.

The compound (1) used in the present invention is the compound of the formula (1):

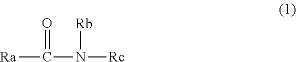

wherein Ra is the formula (2):

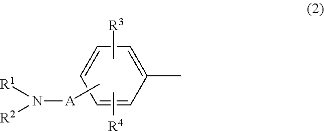

wherein R¹ is a hydrogen, an alkyl, or a cycloalkyl, a cycloalkylalkyl, a phenyl or an aralkyl, which optionally has a substituent on a ring, or a group of the formula (3):

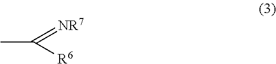

wherein R⁶ is hydrogen, alkyl or the formula: —NR⁸R⁹ wherein R⁸ and R⁹ are the same or different and each is hydrogen, alkyl, aralkyl or phenyl, and R⁷ is hydrogen, alkyl, aralkyl, phenyl, nitro or cyano, or R⁶ and R⁷ combinedly form a heterocycle optionally having oxygen atom, sulfur atom or optionally substituted nitrogen atom additionally in the ring; R² is a hydrogen, an alkyl, or a cycloalkyl, a cycloalkylalkyl, a phenyl or an aralkyl, which optionally has a substituent on the ring; or
R¹ and R² combinedly form, together with the adjacent nitrogen atom, a heterocycle optionally having oxygen atom, sulfur atom or optionally substituted nitrogen atom additionally in the ring;
R³ and R⁴ are the same or different and each is a hydrogen, an alkyl, an aralkyl, a halogen, a nitro, an amino, an alkylamino, an acylamino, a hydroxy, an alkoxy, an aralkyloxy, a cyano, an acyl, a mercapto, an alkylthio, an aralkylthio, a carboxy, an alkoxycarbonyl, a carbamoyl, an alkylcarbamoyl or an azide; A is the formula (4):

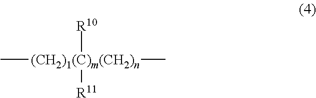

wherein R¹⁰ and R¹¹ are the same or different and each is hydrogen, alkyl, haloalkyl, aralkyl, hydroxyalkyl, carboxy or alkoxycarbonyl, or
R¹⁰ and R¹¹ combinedly form cycloalkyl, and l, m and n are each
0 or an integer of 1-3;
Rb is a hydrogen or an alkyl; and
Rc is an optionally substituted heterocycle containing nitrogen,
or a pharmaceutically acceptable salt thereof.

Each symbol in the present specification means the following.

Alkyl at R¹ and R² is straight or branched alkyl having 1 to 6 carbon atoms, and exemplified by methyl, ethyl, propyl, isopropyl, butyl, isobutyl, sec-butyl, tert-butyl, pentyl, hexyl and the like, with preference given to alkyl having 1 to 4 carbon atoms.

Cycloalkyl at R¹ and R² is cycloalkyl having 3 to 7 carbon atoms such as cyclopropyl, cyclobutyl, cyclopentyl, cyclohexyl and cycloheptyl.

Cycloalkylalkyl at R¹ and R² is that having, as a cycloalkyl moiety, the aforementioned cycloalkyl having 3 to 7 carbon atoms and straight or branched alkyl having 1 to 6 carbon atoms (e.g., methyl, ethyl, propyl, isopropyl, butyl, pentyl and hexyl) as an alkyl moiety, and exemplified by cyclopropylmethyl, cyclobutylmethyl, cyclopentylmethyl, cyclohexylmethyl, cycloheptylmethyl, cyclopropylethyl, cyclopentylethyl, cyclohexylethyl, cycloheptylethyl, cyclopropylpropyl, cyclopentylpropyl, cyclohexylpropyl, cycloheptylpropyl, cyclopropylbutyl, cyclopentylbutyl, cyclohexylbutyl, cycloheptylbutyl, cyclopropyihexyl, cyclopentylhexyl, cyclohexylhexyl, cycloheptylhexyl and the like.

Aralkyl at R¹ and R² is that having, as an alkyl moiety, alkyl having 1 to 4 carbon atoms, and is exemplified by phenylalkyl such as benzyl, 1-phenylethyl, 2-phenylethyl, 3-phenylpropyl and 4-phenylbutyl.

The substituent of cycloalkyl, cycloalkylalkyl, phenyl and aralkyl which may have substituent on the ring at R¹ and R² is halogen (e.g., chlorine, bromine, fluorine and iodine), alkyl (same as alkyl at R¹ and R²), alkoxy (straight or branched alkoxy having 1 to 6 carbon atoms, such as methoxy, ethoxy, propoxy, isopropoxy, butoxy, isobutoxy, sec-butoxy, tert-butoxy, pentyloxy and hexyloxy), aralkyl (same as aralkyl at R¹ and R²), haloalkyl (alkyl at R¹ and R² substituted by 1 to 5 halogen(s), such as fluoromethyl, difluoromethyl, trifluoromethyl, 2,2,2-trifluoroethyl and 2,2,3,3,3-pentafluoropropyl), nitro, amino, cyano, azide and the like.

The heterocycle formed by R¹ and R² in combination together with the adjacent nitrogen atom, which optionally has oxygen atom, sulfur atom or optionally substituted nitrogen atom additionally in the ring is preferably 5 or 6-membered ring or a ring bonded thereto. Specific examples include 1-pyrrolidinyl, piperidino, 1-piperazinyl, morpholino, thiomorpholino, 1-imidazolyl, 2,3-dihydrothiazol-3-yl and the like. The substituent at optionally substituted nitrogen atom is exemplified by alkyl, aralkyl, haloalkyl and the like, wherein alkyl, aralkyl and haloalkyl are the same as those defined for R¹ and R².

Halogen, alkyl, alkoxy and aralkyl at R³ and R⁴ are the same as those exemplified for R¹ and R².

Acyl at R³ and R⁴ is, for example, alkanoyl having 2 to 6 carbon atoms (e.g., acetyl, propionyl, butyryl, valeryl and pivaloyl), benzoyl, or phenylalkanoyl whose alkanoyl moiety has 2 to 4 carbon atoms (e.g., phenylacetyl, phenylpropionyl and phenylbutyryl).

Alkylamino at R³ and R⁴ is that having, at an alkyl moiety, straight or branched alkyl having 1 to 6 carbon atoms, and exemplified by methylamino, ethylamino, propylamino, isopropylamino, butylamino, isobutylamino, sec-butylamino, tert-butylamino, pentylamino, hexylamino and the like.

Acylamino at R³ and R⁴ is that having, as acyl, alkanoyl having 2 to 6 carbon atoms, benzyl, or phenylalkanoyl whose alkanoyl moiety has 2 to 4 carbon atoms, and exemplified by acetylamino, propionylamino, butyrylamino, valerylamino, pivaloylamino, benzoylamino, phenylacetylamino, phenylpropionylamino, phenylbutyrylamino and the like.

Alkylthio at R³ and R⁴ is that having, at an alkyl moiety, straight or branched alkyl having 1 to 6 carbon atoms, and exemplified by methylthio, ethylthio, propylthio, isopropylthio, butylthio, isobutylthio, sec-butylthio, tert-butylthio, pentylthio, hexylthio and the like.

Aralkyloxy at R³ and R⁴ is that including aralkyl having, as an alkyl moiety, alkyl having 1 to 4 carbon atoms, and exemplified by benzyloxy, 1-phenylethyloxy, 2-phenylethyloxy, 3-phenylpropyloxy, 4-phenylbutyloxy and the like.

Aralkylthio at R³ and R⁴ is that including aralkyl having, as an alkyl moiety, alkyl having 1 to 4 carbon atoms, and exemplified by benzylthio, 1-phenylethylthio, 2-phenylethylthio, 3-phenylpropylthio, 4-phenylbutylthio and the like.

Alkoxycarbonyl at R³ and R⁴ is that having, at an alkoxy moiety, straight or branched alkoxy having 1 to 6 carbon atoms, and exemplified by methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, hexyloxycarbonyl and the like.

Alkylcarbamoyl at R³ and R⁴ is carbamoyl mono- or di-substituted by alkyl having 1 to 4 carbon atoms, and exemplified by methylcarbamoyl, dimethylcarbamoyl, ethylcarbamoyl, diethylcarbamoyl, propylcarbamoyl, dipropylcarbamoyl, butylcarbamoyl, dibutylcarbamoyl and the like.

Alkyl at Rb is the same as alkyl at R¹ and R².

Heterocycle containing nitrogen at Rc when it is monocyclic is, for example, pyridine, pyrimidine, pyridazine, triazine, pyrazole or triazole, and when it is a condensed ring, it is exemplified by pyrrolopyridine (e.g., 1H-pyrrolo[2,3-b]pyridine, 1H-pyrrolo[3,2-b]pyridine and 1H-pyrrolo[3,4-b]pyridine), pyrazolopyridine (e.g., 1H-pyrazolo[3,4-b]pyridine and 1H-pyrazolo[4,3-b]pyridine), imidazopyridine (e.g., 1H-imidazo[4,5-b]pyridine), pyrrolopyrimidine (e.g., 1H-pyrrolo[2,3-d]pyrimidine, 1H-pyrrolo[3,2-d]pyrimidine and 1H-pyrrolo[3,4-d]pyrimidine), pyrazolopyrimidine (e.g., 1H-pyrazolo[3,4-d]pyrimidine, pyrazolo[1,5-a]pyrimidine and 1H-pyrazolo[4,3-d]pyrimidine), imidazopyrimidine (e.g., imidazo[1,2-a]pyrimidine and 1H-imidazo[4,5-d]pyrimidine), pyrrolotriazine (e.g., pyrrolo[1,2-a]-1,3,5-triazine and pyrrolo[2,1-f]-1,2,4-triazine), pyrazolotriazine (e.g., pyrazolo[1,5-a]-1,3,5-triazine), triazolopyridine (e.g., 1H-1,2,3-triazolo[4,5-b]pyridine), triazolopyrimidine (e.g., 1,2,4-triazolo[1,5-a]pyrimidine, 1,2,4-triazolo[4,3-a]pyrimidine and 1H-1,2,3-triazolo[4,5-d]pyrimidine), cinnoline, quinazoline, quinoline, pyridopyridazine (e.g., pyrido[2,3-c]pyridazine), pyridopyrazine (e.g., pyrido[2,3-b]pyrazine), pyridopyrimidine (e.g., pyrido[2,3-d]pyrimidine and pyrido[3,2-d]pyrimidine), pyrimidopyrimidine (e.g., pyrimido[4,5-d]pyrimidine and pyrimido[5,4-d]pyrimidine), pyrazinopyrimidine (e.g., pyrazino[2,3-d]pyrimidine), naphthylidine (e.g., 1,8-naphthylidine), tetrazolopyrimidine (e.g., tetrazolo[1,5-a]pyrimidine), thienopyridine (e.g., thieno[2,3-b]pyridine), thienopyrimidine (e.g., thieno[2,3-d]pyrimidine), thiazolopyridine (e.g., thiazolo[4,5-b]pyridine and thiazolo[5,4-b]pyridine), thiazolopyrimidine (e.g., thiazolo[4,5-d]pyrimidine and thiazolo[5,4-d]pyrimidine), oxazolopyridine (e.g., oxazolo[4,5-b]pyridine and oxazolo[5,4-b]pyridine), oxazolopyrimidine (e.g., oxazolo[4,5-d]pyrimidine and oxazolo[5,4-d]pyrimidine), furopyridine (e.g., furo[2,3-b]pyridine and furo[3,2-b]pyridine), furopyrimidine (e.g., furo[2,3-d]pyrimidine and furo[3,2-d]pyrimidine), 2,3-dihydropyrrolopyridine (e.g., 2,3-dihydro-1H-pyrrolo[2,3-b]pyridine and 2,3-dihydro-1H-pyrrolo[3,2-b]pyridine), 2,3-dihydropyrrolopyrimidine (e.g., 2,3-dihydro-1H-pyrrolo[2,3-d]pyrimidine and 2,3-dihydro-1H-pyrrolo[3,2-d]pyrimidine), 5,6,7,8-tetrahydropyrido[2,3-d]pyrimidine, 5,6,7,8-tetrahydro-1,8-naphthylidine, 5,6,7,8-tetrahydroquinoline and the like. When these rings form hydrogenated aromatic rings, the carbon atom in the ring may be carbonyl. Examples thereof include 2,3-dihydro-2-oxopyrrolopyridine, 2,3-dihydro-2,3-dioxopyrrolopyridine, 7,8-dihydro-7-oxo-1,8-naphthylidine, 5,6,7,8-tetrahydro-7-oxo-1,8-naphthylidine and the like.

These rings may be substituted by substituent such as halogen, alkyl, alkoxy, aralkyl, haloalkyl, nitro, amino, alkylamino, cyano, formyl, acyl, aminoalkyl, mono- or dialkylaminoalkyl, azide, carboxy, alkoxycarbonyl, carbamoyl, alkylcarbamoyl, optionally substituted hydrazino and the like.

The substituent of optionally substituted hydrazino include, for example, alkyl, aralkyl, nitro and cyano, wherein alkyl and aralkyl are the same as alkyl and aralkyl at R¹ and R², and optionally substituted hydrazino is exemplified by methylhydrazino, ethylhydrazino, benzylhydrazino, and the like.

Alkyl at R⁶ is the same as alkyl at R¹ and R²; alkyl at R⁸ and R⁹ is the same as alkyl at R¹ and R²; and aralkyl at R⁸ and R⁹ is the same as aralkyl at R¹ and R².

Alkyl at R⁷ is the same as alkyl at R¹ and R², and aralkyl at R⁷ is the same as alkyl at R¹ and R².

The group formed combinedly by R⁶ and R⁷, which forms a heterocycle optionally having oxygen atom, sulfur atom or optionally substituted nitrogen atom additionally in the ring may be, for example, imidazol-2-yl, thiazol-2-yl, oxazol-2-yl, imidazolin-2-yl, 3,4,5,6-tetrahydropyridin-2-yl, 3,4,5,6-tetrahydropyrimidin-2-yl, 1,3-oxazolin-2-yl, 1,3-thiazolin-2-yl, or benzimidazol-2-yl, benzothiazol-2-yl or benzoxazol-2-yl which may have substituent such as halogen, alkyl, alkoxy, haloalkyl, nitro, amino, phenyl, aralkyl and the like. By halogen, alkyl, alkoxy, haloalkyl and aralkyl are meant those exemplified for R¹ and R².

The substituent of the above-mentioned optionally substituted nitrogen atom may be, for example, alkyl, aralkyl or haloalkyl, wherein alkyl, aralkyl and haloalkyl are those exemplified for R¹ and R².

Hydroxyalkyl at R¹⁰ and R¹¹ is straight or branched alkyl having 1 to 6 carbon atoms, which is substituted by 1 to 3 hydroxy, such as hydroxymethyl, 2-hydroxyethyl, 1-hydroxyethyl, 3-hydroxypropyl and 4-hydroxybutyl. Alkyl at R¹⁰ and R¹¹ is the same as those at R¹ and R²; haloalkyl and alkoxycarbonyl at R¹⁰ and R¹¹ are the same as those at R¹ and R²; and aralkyl at R¹⁰ and R¹¹ is the same as those at R¹ and R². Cycloalkyl combinedly formed by R¹⁰ and R¹¹ is the same as cycloalkyl at R¹ and R².

Compound (1) is preferably (R)-(+)-N-(1H-pyrrolo[2,3-b]pyridin-4-yl)-4-(1-aminoethyl)benzamide or a pharmacologically acceptable salt thereof. In the following, for convenience, (R)-(+)-N-(1H-pyrrolo[2,3-b]pyridin-4-yl)-4-(1-aminoethyl)benzamide or a pharmacologically acceptable salt thereof is sometimes referred to as compound (Ia). As the salt of the compound, a pharmaceutically acceptable acid addition salt is preferable. Examples of the acid include inorganic acids such as hydrochloric acid, hydrobromic acid, sulfuric acid and the like, organic acids such as methanesulfonic acid, fumaric acid, maleic acid, mandelic acid, citric acid, tartaric acid, salicylic acid and the like. Of these, (R)-(+)-N-(1H-pyrrolo[2,3-b]pyridin-4-yl)-4-(1-aminoethyl)benzamide monohydrochloride (hereinafter to be sometimes referred to as compound (I)) is preferable.

Compound (Ia) may be a hydrate, and 1 hydrate, 2 hydrate, ½ hydrate, ⅓ hydrate, ¼ hydrate, ⅔ hydrate, 3/2 hydrate, 6/5 hydrate and the like of compound (Ia) are also encompassed in the present invention.

Compound (1), specifically compound (I), can be specifically synthesized by, for example, the methods described in W)95/28387 and WO2002/083175.

Compound (1), preferably compound (Ia), particularly preferably compound (I), and pharmacologically acceptable salts thereof, and hydrates thereof to be used in the present invention are also referred to as the compound of the present invention.

In the present specification, the “corneal endothelial dysfunction” refers to a state where corneal endothelial cells are damaged or impaired for some cause. Examples of the cause include intraocular surgery, increased intraocular pressure, contact lense wearing and the like.

In the present specification, the “treatment of corneal endothelial dysfunction” is a concept including not only the treatment of corneal endothelial dysfunction but also the prophylaxis of the dysfunction. In addition, the “corneal endothelial dysfunction” also includes “a disease associated with corneal endothelial dysfunction”. Examples of the disease include bullous keratopathy, corneal endotheliitis, corneal edema, corneal leukoma and the like, and the present invention can be appropriately applied thereto as target diseases according to the embodiment of the present invention.

Corneal endothelial cells play a role of maintaining transparency of the cornea. When the density of corneal endothelial cells decreases over a certain limit, the cornea develops swelling and becomes incapable of maintaining transparency, whereby corneal endothelial dysfunction is developed. The therapeutic agent of the present invention promotes adhesion of corneal endothelial cells, and can form the corneal endothelial cell layer having good cell morphology, normal function and high cell density. Furthermore, the therapeutic agent of the present invention suppresses apoptosis of corneal endothelial cells and can treat or prevent corneal endothelial dysfunction. The therapeutic agent of the present invention can treat or prevent a disease associated with corneal endothelial dysfunction, for example, bullous keratopathy and corneal endotheliitis. In addition, the therapeutic agent of the present invention can treat or prevent corneal endothelial dysfunction caused by intraocular surgery such as cataract surgery, vitreous surgery and the like, corneal endothelial dysfunction caused by increased intraocular pressure (particularly glaucomatous attack) or corneal endothelial dysfunction caused by less oxygen due to contact lenses worn.

The therapeutic agent of the present invention is not particularly limited as long as it has a dosage form suitable for topical administration to the eye and, for example, the forms of intracameral injection, intraocular irrigating solution, eye drop and the like can be mentioned. In the present invention, preferred are intraocular irrigating solution or eye drop, and more preferred is eye drop from the aspect of easy administration. They can be prepared using conventional techniques widely used in the field. When topically administered to the eye in the form of an intracameral injection or intraocular irrigating solution, the compound of the present invention comes into contact with corneal endothelial cells in vivo, and healing of corneal endothelial wound is promoted. In the case of eye drop, the compound of the present invention reaches corneal endothelial cells from cornea epithelium through corneal stroma. A part thereof transfers into aqueous humor, contacts corneal endothelial cells from the aqueous humor side, and promotes healing of corneal endothelial wound.

In the therapeutic agent of the present invention, stabilizer (e.g., sodium bisulfite, sodium thiosulfate, sodium edetate, sodium citrate, ascorbic acid, dibutylhydroxytoluene etc.), solubilizer (e.g., glycerol, propylene glycol, macrogol, polyoxyethylene hydrogenated castor oil, polysorbate 80 etc.), suspending agent (e.g., polyvinylpyrrolidone, hydroxypropylmethylcellulose, hydroxymethylcellulose, carboxymethylcellulose sodium etc.), emulsifier (e.g., polyvinylpyrrolidone, soybean lecithin, egg-yolk lecithin, polyoxyethylene hydrogenated castor oil, polysorbate 80 etc.), buffer agent (e.g., phosphate buffer, acetate buffer, borate buffer, carbonate buffer, citrate buffer, Tris buffer, glutamic acid, epsilon-aminocaproic acid etc.), thickening agent (e.g., water-soluble cellulose derivative such as methylcellulose, hydroxyethylcellulose, hydroxypropylmethylcellulose, carboxymethylcellulose etc., sodium chondroitin sulfate, sodium hyaluronate, carboxyvinyl polymer, polyvinyl alcohol, polyvinylpyrrolidone, macrogol etc.), preservative (e.g., benzalkonium chloride, benzethonium chloride, chlorhexidine gluconate, chlorobutanol, benzyl alcohol, sodium dehydroacetate, p-hydroxybenzoic esters, sodium edetate, boric acid etc.), isotonicity agent (e.g., sodium chloride, potassium chloride, glycerol, mannitol, sorbitol, boric acid, glucose, propylene glycol etc.), pH adjuster (e.g., hydrochloric acid, sodium hydroxide, phosphoric acid, acetic acid etc.), algefacient (e.g., 1-menthol, d-camphor, d-borneol, peppermint oil etc.) and the like can be added as additives. The amount of these additives to be added varies depending on the kind and use of the additive, and the like, and may be added at a concentration capable of achieving the object of the additive.

While the amount of the active ingredient in the therapeutic agent of the present invention varies depending on the kind of the compound of the present invention to be used, the amount of compound (Ia) or compound (I) is generally about 0.00001-1 w/v %, preferably about 0.00001-0.1 w/v %, more preferably about 0.0001-0.05 w/v %, about 0.001-0.05 w/v %, about 0.002-0.05 w/v %, about 0.003-0.05 w/v %, about 0.004-0.05 w/v %, about 0.005-0.05 w/v %, about 0.006-0.05 w/v %, about 0.007-0.05 w/v %, about 0.008-0.05 w/v %, about 0.009-0.05 w/v %, about 0.01-0.05 w/v %, about 0.02-0.05 w/v %, about 0.03-0.05 w/v %, about 0.04-0.05 w/v %, about 0.003-0.04 w/v %, about 0.004-0.04 w/v %, about 0.005-0.04 w/v %, about 0.006-0.04w/v %, about 0.007-0.04 w/v %, about 0.008-0.04 w/v %, about 0.009-0.04 w/v %, about 0.01-0.04w/v %, about 0.02-0.04 w/v %, about 0.03-0.04 w/v %, about 0.003-0.03 w/v %, about 0.004-0.03 w/v %, about 0.005-0.03 w/v %, about 0.006-0.03 w/v %, about 0.007-0.03 w/v %, about 0.008-0.03 w/v %, about 0.009-0.03 w/v %, about 0.01-0.03 w/v %, about 0.02-0.03 w/v %, about 0.003-0.02 w/v %, about 0.004-0.02 w/v %, about 0.005-0.02 w/v %, about 0.006-0.02 w/v %, about 0.007-0.02 w/v %, about 0.008-0.02 w/v %, about 0.009-0.02 w/v %, about 0.01-0.02 w/v %, about 0.003-0.01 w/v %, about 0.004-0.01 w/v %, about 0.005-0.01 w/v %, about 0.006-0.01 w/v %, about 0.007-0.01 w/v %, about 0.008-0.01 w/v % or about 0.009-0.01 w/v %. While the dose and administration frequency vary depending on the symptom, age, body weight and administration form, when it is used as an eye drop to an adult, for example, a preparation containing about 0.0001-0.1 w/v %, preferably about 0.003-0.03 w/v %, of an active ingredient can be generally administered 1-10 times, preferably 1-6 times, more preferably 1-3 times, per day, by about 0.01-0.1 mL per administration. When the therapeutic agent of the present invention is injected into the anterior chamber, a concentration of 1/10- 1/1000 of the above-mentioned concentration can be used. In the therapeutic agent of the present invention, those of ordinary skill in the art can appropriately determine the concentration of the compound of the present invention depending on the disease condition.

Examples of the subject of administration of the therapeutic agent of the present invention include mammals (e.g., human, mouse, rat, hamster, rabbit, cat, dog, bovine, horse, sheep, monkey etc.).

In the present specification, the “promotion of adhesion of corneal endothelial cell” refers to the promoting the adhesion of corneal endothelial cells. Examples of the promotion of adhesion of corneal endothelial cells include promotion of adhesion between corneal endothelial cells, promotion of adhesion of corneal endothelial cells to Descemet's membrane, and promotion of adhesion of corneal endothelial cells to a culture substrate or scaffold.

In the present specification, the “agent for promoting adhesion” is a medicament having an action to promote adhesion. The agent for promoting adhesion of corneal endothelial cells of the present invention (hereinafter to be sometimes abbreviated as “agent for promoting adhesion of the present invention”) has an action to promote adhesion of corneal endothelial cells separated from a corneal tissue derived from a mammal, adhesion between corneal endothelial cells separated therefrom and passaged, adhesion of corneal endothelial cells to Descemet's membrane, and adhesion of corneal endothelial cells to a culture substrate or scaffold, wherein the mammal includes, for example, human, mouse, rat, hamster, rabbit, cat, dog, bovine, horse, sheep, monkey and the like. Since the agent for promoting adhesion of the present invention is superior in an adhesion promoting action of corneal endothelial cells derived from human, which are considered to be particularly difficult to culture and passage, human-derived corneal endothelial cells are a preferable target.

The agent for promoting adhesion of the present invention can be used as an agent for protecting corneal endothelium in the treatment or prophylaxis of diseases associated with corneal endothelial dysfunction. Examples of the disease associated with corneal endothelial dysfunction include bullous keratopathy, corneal endotheliitis and the like. In addition, the agent for promoting adhesion of the present invention can also be used as an agent for protecting corneal endothelium in the treatment or prophylaxis of corneal endothelial dysfunction caused by intraocular surgery such as cataract surgery, vitreous surgery and the like, corneal endothelial dysfunction caused by increased intraocular pressure (particularly glaucomatous attack), or corneal endothelial dysfunction caused by less oxygen due to contact lenses worn. Without particular limitation, the agent for promoting adhesion of the present invention can contain an additive (stabilizer, solubilizer, suspending agent etc.) similar to those used for the above-mentioned therapeutic agent. The content, dose, subject of administration and the like of the compound of the present invention as an active ingredient can also be similar to those for the above-mentioned therapeutic agent.

The agent for promoting adhesion of the present invention can also be added to a culture medium when corneal endothelial cells are cultured in vitro. When the compound of the present invention is added to the culture medium and culture is continued, the compound of the present invention contacts corneal endothelial cells and adhesion between corneal endothelial cells, adhesion of corneal endothelial cells to

Descemet's membrane, and adhesion of corneal endothelial cells to a culture substrate or scaffold are promoted. In the case of adding the agent for promoting adhesion of the present invention to the culture medium, while the concentration of the compound of the present invention, and the like are contained in the culture medium can be similar to that of below-mentioned present invention, it is not particularly limited to this.

In other aspect, the present invention provides culture medium of corneal endothelial cells containing the compound of the present invention. The compound of the present invention contained in the culture medium of the present invention is as described above.

Without particular limitation, the culture medium of the present invention can contain a medium generally used for culture of corneal endothelial cells (e.g., Dulbecco's Modified Eagle Medium (DMEM, Invitrogen), serum (e.g., fetal bovine serum (FBS)), growth factors (e.g., basic-fibroblast growth factor (b-FGF)), antibiotics (e.g., penicillin, streptomycin) and the like.

In the culture medium of the present invention, while the concentration of the compound of the present invention varies depending on a kind of compound to be used, the case of compound (Ia) or compound (I) is generally about 0.001-100 μM, preferably, about 0.01-75 μM, about 0.05-50 μM, about 1-10 μM, about 0.01-10 μM, about 0.05-10 μM, about 0.075 5-10 μM, about 0.1-10 μM, about 0.5-10 μM, about 0.75-10 μM, about 1.0-10 μM, about 1.25-10 μM, about 1.5-10 μM, about 1.75-10 μM, about 2.0-10 μM, about 2.5-10 μM, about 3.0-10 μM, about 4.0-10 μM, about 5.0-10 μM, about 6.0-10 μM, about 7.0-10 μM, about 8.0-10 μM, about 9.0-10 μM, about 0.01-5.0 μM, about 0.05-5.0 μM, about 0.075-5.0 μM, about 0.1-5.0 μM, about 0.5-5.0 μM, about 0.75-5.0 μM, about 1.0-5.0 μM, about 1.25-5.0 μM, about 1.5-5.0 μM, about 1.75-5.0 μM, about 2.0-5.0 μM, about 2.5-5.0 μM, about 3.0- 5.0 μM, about 4.0-5.0 μM, about 0.01-3.0 μM, about 0.05-3.0 μM, about 0.075-3.0 μM, about 0.1-3.0 μM, about 0.5-3.0 μM, about 0.75-3.0 μM, about 1.0-3.0 μM, about 1.25-3.0 μM, about 1.5-3.0 μM, about 1.75-3.0 μM, about 2.0-3.0 μM, about 0.01-1.0 μM, about 0.05-1.0 μM, about 0.075-1.0 μM, about 0.1-1.0 μM, about 0.5-1.0 μM, about 0.75-1.0 μM, about 0.09-3.5 μM, about 0.09-3.2 μM, more preferably, about 0.05-1.0 μM, about 0.075-1.0 μM, about 0.1-1.0 μM, about 0.5-1.0 μM, about 0.75-1.0 μM.

The culture medium of the present invention prevents dissociation of the cells by promoting adhesion of corneal endothelial cells, and enables formation of the corneal endothelial cell layer having good cell morphology, normal function and high cell density. Therefore, it is preferably used for the production method of the corneal endothelial preparation of the present invention mentioned below. In addition, the culture medium of the present invention is also used for maintaining corneal endothelial cells.

In other aspect, the present invention provides a corneal storage solution containing the compound of the present invention. The compound of the present invention contained in the corneal storage solution of the present invention is as described above. In the present description, a corneal storage solution is a liquid used for preserving a corneal graft isolated from a donor until transplantation to a recipient.

Examples of the corneal storage solution of the present invention include storage solutions generally used for corneal endothelial keratoplasty (corneal storage media (Optisol GS:

registered trade mark), eye storage medium (EPII: registered trade mark)), saline, phosphate buffered saline (PBS) and the like, each of which contains the compound of the present invention.

In the corneal storage solution of the present invention, the concentration of the compound of the present invention varies depending on the kind of the compound to be used. The concentration of compound (Ia) or compound (I) is generally about 0.001-100 μM, preferably about 0.01-75 μM, about 0.05-50 μM, about 1-10 μM, about 0.01-10 μM, about 0.05- 10 μM, about 0.075-10 μM, about 0.1-10 μM, about 0.5-10 μM, about 0.75-10 μM, about 1.0-10 μM, about 1.25-10 μM, about 1.5-10 μM, about 1.75-10 μM, about 2.0-10 μM, about 2.5-10 μM, about 3.0-10 μM, about 4.0-10 μM, about 5.0-10 μM, about 6.0-10 μM, about 7.0-10 μM, about 8.0-10 μM, about 9.0-10 μM, about 0.01-5.0 μM, about 0.05-5.0 μM, about 0.075-5.0 μM, about 0.1-5.0 μM, about 0.5-5.0 μM, about 0.75-5.0 μM, about 1.0-5.0 μM, about 1.25-5.0 μM, about 1.5-5.0 μM, about 1.75-5.0 μM, about 2.0-5.0 μM, about 2.5-5.0 μM, about 3.0-5.0 μM, about 4.0-5.0 μM, about 0.01-3.0 μM, about 0.05-3.0 μM, about 0.075- 3.0 μM, about 0.1-3.0 μM, about 0.5-3.0 μM, about 0.75-3.0 μM, about 1.0-3.0 μM, about 1.25-3.0 μM, about 1.5-3.0 μM, about 1.75-3.0 μM, about 2.0-3.0 μM, about 0.01-1.0 μM, about 0.05-1.0 μM, about 0.075-1.0 μM, about 0.1-1.0 μM, about 0.5-1.0 μM, about 0.75-1.0 μM, about 0.09-3.5 μM or about 0.09-3.2 μM, more preferably about 0.05-1.0 μM, about 0.075-1.0 μM, about 0.1-1.0 μM, about 0.5-1.0 μM or about 0.75-1.0 μM.

The corneal storage solution of the present invention prevents dissociation of the cells by promoting adhesion of corneal endothelial cells, and enables formation of the corneal endothelial cell layer having good cell morphology, normal function and high cell density. Therefore, it is preferably used as a preservation solution of a cornea to be used for organ transplantation and the like. The corneal storage solution of the present invention provides an effect of suppressing cell death and apoptosis of corneal endothelial cells in preservation. In addition, the corneal storage solution of the present invention is also used as a storage solution for cryopreservation of corneal endothelial cells. For cryopreservation, glycerol, dimethyl sulfoxide, propylene glycol, acetamide and the like may be further added to the corneal storage solution of the present invention.

In one aspect, the present invention provides a corneal endothelial preparation containing the compound of the present invention and corneal endothelial cells. In the present specification, the “corneal endothelial preparation” refers to a preparation that prevents, reduces or disappears the condition of corneal endothelial dysfunction.

The corneal endothelial preparation of the present invention can treat a disease having a disorder in the corneal endothelium, as long as it contains corneal endothelial cells and the compound of the present invention. Not bound by theory, it is because when the compound of the present invention contacts corneal endothelial cells in vivo, adhesion of the corneal endothelial cells to the Descemet's membrane is promoted. In addition, it is considered that since the compound of the present invention promotes re-adhesion of the cells dissociated during transplantation to the Descemet's membrane and suppresses cell apoptosis, healing of corneal endothelial wound can be promoted. Any preparations that exhibit a treatment effect conveniently and soon, like the corneal endothelial preparation of the present invention, have not existed heretofore, and they are provided for the first time by the present invention.

In one embodiment, the corneal endothelial cells to be contained in the corneal endothelial preparation of the present invention may be ones cultured in a culture medium containing the compound of the present invention, or a culture medium not containing the compound of the present invention. In other embodiment, in the corneal endothelial preparation of the present invention, the compound of the present invention and corneal endothelial cells may be mixed immediately before administration, or preserved as a mixture. In another embodiment, the-corneal endothelial preparation of the present invention may contain the culture medium or a corneal storage solution of the present invention, or both, so as to maintain the corneal endothelial cells. In another embodiment, the corneal endothelial preparation of the present invention may contain a solution to suspend the corneal endothelial cells. As mentioned above, when the compound of the present invention and corneal endothelial cells are present in the site to be treated and come into contact with each other, healing of the corneal endothelial wound is promoted.

In a preferable embodiment, the corneal endothelial preparation of the present invention may contain compound (Ia) ((R)-(+)-N-(1H-pyrrolo[2,3-b]pyridin-4-yl)-4-(1-aminoethyl)benzamido or a pharmacologically acceptable salt thereof) as the compound of the present invention.

The corneal endothelial preparation of the present invention can be used for the treatment of diseases associated with corneal endothelial dysfunction, for example, bullous keratopathy, corneal endotheliitis, corneal edema and corneal leukoma, particularly, bullous keratopathy caused by corneal endothelial dysfunction due to corneal dystrophy, trauma or intraocular surgery. In addition, the corneal endothelial preparation of the present invention can be directly administered into the anterior chamber of patient having a disease with a disorder in the corneal endothelium by, for example, injection and the like.

The compound of the present invention, corneal endothelial cells and the like to be used for the corneal endothelial preparation of the present invention may be in any form in the above-mentioned therapeutic agent of the present invention. In addition, the amount of the compound of the present invention to be contained in the corneal endothelial preparation of the present invention can also be similar to, but is not limited to, for example, the content in the above-mentioned therapeutic agent. The amount can be appropriately determined according to the embodiment of the corneal endothelial preparation.

In other aspect, the present invention provides a production method of a corneal endothelial preparation, comprising a step of culturing corneal endothelial cells using the culture medium containing the compound of the present invention, and a corneal endothelial preparation obtained by the production method. The compound of the present invention, corneal endothelial cells and the like to be used for the production method and the corneal endothelial preparation of the present invention may be in any form mentioned above.

In one embodiment, the production method of the present invention includes a step of culturing corneal endothelial cells using the culture medium of the present invention and, for example, can be performed by the following method.

### <1> Collection of Corneal Endothelial Cells and Culture in Vitro

Corneal endothelial cells are collected from the cornea of the recipient himself/herself or a suitable donor by a conventional method. In consideration of the transplantation conditions in the present invention, allogeneic corneal endothelial cells may be prepared. For example, Descemet's membrane is stripped together with intact corneal endothelial cells and treated with collagenase and the like. After isolation of corneal endothelial cells, the corneal endothelial cells are cultured in the culture medium of the present invention. A culture medium can be used, for example, by appropriately adding FBS (fetal bovine serum), basic-fibroblast growth factor (b-FGF), and antibiotics such as penicillin, streptomycin and the like to commercially available Dulbecco's Modified Eagle's Medium (DMEM), and further adding the compound of the present invention, preferably compound (Ia), thereto. A culture flask (culture dish) with a coating of type I collagen, type IV collagen, fibronectin, laminin or extracellular matrix of bovine corneal endothelial cells, and the like on the surface is preferably used. Alternatively, a general culture flask treated with a commercially available coating agent such as FNC coating mix (registered trade mark) and the like may be used. By a combined use of such coating and the culture medium of the present invention, adhesion of corneal endothelial cells to the surface of a culture flask is promoted, and good growth is made.

While the temperature conditions for culture of corneal endothelial cells are not particularly limited as long as the corneal endothelial cells grow, for example, the temperature is about 25- about 45° C., preferably about 30- about 40° C. in consideration of the growth efficiency, and further preferably about 37° C. The culture method is performed in a conventional cell culture incubator with a humidified atmosphere under about 5-10% CO₂ concentration.

### <2> Passage Culture

Passage culture can be performed after growth of the corneal endothelial cells subjected to culture. Passage culture is preferably performed when the cells have reached subconfluent or confluent. Passage culture can be performed as follows. The cells are treated with trypsin-EDTA etc., and collected. The culture medium of the present invention is added to the collected cells to give a cell suspension. A centrifugation treatment is preferably performed during collect of the cells or after collect. Such a centrifugation treatment affords a cell suspension having a high cell density. For example, the cell density of the cell suspension is about 1—2×10⁶ cells/mL. As the conditions for the centrifugation treatment here, for example, 500 rpm (×30 g)—1000 rpm (×70 g), 1-10 min can be mentioned.

The cell suspension is seeded on a culture flask in the same manner as in the above-mentioned primary culture, and cultured. While the dilution ratio during passage varies depending on the condition of cells, it is about 1:2 -1:4, preferably about 1:3. The passage culture can be performed under culture conditions similar to those of the above-mentioned primary culture. While the culture time varies depending on the condition of cells to be used, it is, for example, 7 -30 days. The above passage culture can be performed multiple times as necessary. Using the culture medium of the present invention, cell adhesion in the early stages of culture is increased, whereby the culture time can be shortened.

By culturing as mentioned above, a corneal endothelial preparation containing corneal endothelial cells and the compound (preferably, compound (Ia)) of the present invention can be obtained.

In another aspect, the present invention provides a kit for the treatment of corneal endothelial dysfunction. The kit includes the compound of the present invention, corneal endothelial cells and an instruction.

In one embodiment, the compound of the present invention to be contained in the kit of the present invention may be contained in, for example, a washing solution used to wash the corneal endothelial cells, a culture medium in which to cultivate the corneal endothelial cells, a solution for cell suspension to suspend the corneal endothelial cells and the like, or may be in the form of a solid (e.g., powder). It is because when the compound of the present invention and the corneal endothelial cells are present in the site to be treated and come into contact with each other, healing of the corneal endothelial wound is promoted. In a preferable embodiment, the compound of the present invention can be compound (Ia) ((R)-(+)-N-(1H-pyrrolo[2,3-b]pyridin-4-yl)-4-(1-aminoethyl)benzamide or a pharmacologically acceptable salt thereof). In another embodiment, moreover, the corneal endothelial cells contained in the kit of the present invention may be frozen. The compound of the present invention, corneal endothelial cells and the like to be used for the kit of the present invention can be in any form as in the above-mentioned therapeutic agent of the present invention, corneal endothelial preparation and the like.

In other aspect, the present invention provides an implant for corneal endothelial keratoplasty, containing A) a corneal endothelial cells, B) scaffold and C) the compound of the present invention, preferably compound (Ia) ((R)-(+)-N-(1H-pyrrolo[2,3-b]pyridin-4-yl)-4-(1-aminoethyl)benzamide or a pharmacologically acceptable salt thereof).

In the present specification, the “implant for corneal endothelial keratoplasty” means a piece of tissue, cells, composition, medicament and the like of the present invention to be transplanted into the cornea.

In the present specification, the “scaffold” means a material to support cells. The scaffold has a predetermined strength and biocompatibility. When used in the present specification, the scaffold is produced from a biological substance or a substance supplied by the nature, or a naturally occurring substance or a synthetically supplied substance. Specifically, the scaffold can be formed from a substance (noncellular material) other than organic forms (e.g., tissue, cell). The scaffold to be used in the implant of the present invention is not particularly limited as long as it carries a cultured corneal endothelial cell layer, and can maintain the shape in vivo for at least 3 days post-transplantation. In addition, the scaffold may have a role of scaffold for culture of the corneal endothelial cells in vitro, or may have only a role of carrying the corneal endothelial cell layer after culture. Preferably, the scaffold is used for culturing the corneal endothelial cells, and has a role of scaffold directly applicable to transplantation after completion of the culture. In the present invention, the scaffold and the substrate can be used interchangeably.

Examples of the aforementioned scaffold or substrate include, but are not limited to, polymer materials derived from naturally occurring substance such as collagen, gelatin, cellulose and the like, synthetic polymer materials such as polystyrene, polyester, polycarbonate, poly(N-isopropylacrylamide) and the like, biodegradable polymer materials such as polylactic acid, polyglycolic acid and the like, hydroxyapatite, amniotic membrane and the like.

While the shape of the aforementioned scaffold or substrate is not particularly limited as long as it carries a corneal endothelial cell layer and is suitable for transplantation, a sheet form is preferable. When the implant for corneal endothelial keratoplasty of the present invention is a sheet, it can be cut into a size fitting the application site during the transplantation. In addition, it is also possible to roll the sheet small and insert it from the lip of a wound. A specifically preferable example is a round shape covering about 80% of the area of the abnormal corneal endothelium. In addition, it is also preferable to make a slit around the aforementioned round shape so that the sheet can adhere closely to the application site.

In a preferable embodiment, the aforementioned scaffold or substrate is collagen. As the collagen, a collagen sheet described in JP-A-2004-24852 can be preferably used. Such collagen sheet can be prepared, for example, from amniotic membrane according to the method described in JP-A-2004-24852.

The above-mentioned corneal endothelial cell layer preferably has at least one of the following characteristics. More preferably, it has two or more, and still more preferably all, of the following characteristics.

(1) The cell layer has a single layer structure. This is one of the physiological characteristics of the corneal endothelial cell in vivo.
(2) The cell layer has a cell density of about 1,000- about 4,000 cells/mm². Particularly, it is preferably about 2,000- about 3,000 cells/mm² when the recipient is an adult.
(3) The cell constituting the cell layer forms a hexagonal lattice structure. This is one of physiological characteristics of the cells constituting the corneal endothelial cell layer in vivo. With the hexagonal-shaped cells, the preparation of the present invention can exhibit a function similar to the physiological function of the inherent corneal endothelial cell layer in vivo.
(4) The cells in the cell layer form a cobblestone-like monolayer. Physiologically, the corneal endothelial cells are arranged in the same fashion. This makes possible maintaining corneal normal function and high transparency and regulating corneal hydration appropriately. Therefore, with such morphological characteristics, the implant for corneal endothelial keratoplasty of the present invention is expected to exhibit a function similar to that of the corneal endothelial cell layer in vivo. Since the implant for corneal endothelial keratoplasty of the present invention contains compound (Ia), it can retain corneal endothelial cells well after transplantation.

Preparation of Corneal Endothelial Cell Layer

A cell suspension of the corneal endothelial cells can be prepared according to <1> Collection of corneal endothelial cells and culture in vitro and <2> Passage culture of the above-mentioned corneal endothelial preparation. A cell suspension is seeded on a substrate such as a collagen sheet and the like, and cultured. Here, the number of seeded cells is controlled such that the finally-produced corneal endothelial preparation has a cell layer having a desired cell density. To be precise, cells are seeded such that a cell layer having a cell density of about 1,000 - about 4,000 cells/mm² is formed. Culture can be performed under conditions similar to those of the above-mentioned primary culture and the like. While the culture time varies depending on the condition of cells to be used, it is, for example, 3-30 days. The corneal endothelial cell layer can be prepared in a shorter period while maintaining good morphology and function, by adding the compound of the present invention, preferably compound (Ia) ((R)-(+)-N-(1H-pyrrolo[2,3-b]pyridin-4-yl)-4-(1-aminoethyl)benzamide or a pharmacologically acceptable salt thereof) to a culture medium or cell suspension and the like.

By culturing as mentioned above, the implant for corneal endothelial keratoplasty can be produced as a corneal endothelial cell layer which is cultured on a substrate.

In the present invention, the implant for corneal endothelial keratoplasty may contain the culture medium of the present invention in order to maintain corneal endothelial cell. In addition, the implant for corneal endothelial keratoplasty may contain the corneal storage solution of the present invention until transplantation. The implant for corneal endothelial keratoplasty of the present invention may contain both the culture medium and storage solution of the present invention. In one embodiment, the implant for corneal endothelial keratoplasty of the present invention may further contain at least one selected from a washing solution used to wash the corneal endothelial cells, a culture medium to cultivate the corneal endothelial cells, and a solution to suspend the corneal endothelial cells. As mentioned above, when the compound of the present invention and corneal endothelial cells are present in the site to be treated, and come into contact with each other, healing of the corneal endothelial wound is promoted.

The implant for corneal endothelial keratoplasty of the present invention can be used as a graft for the treatment of a disease requiring corneal endothelial keratoplasty, for example, bullous keratopathy, corneal edema, corneal leukoma, particularly, bullous keratopathy caused by corneal endothelial dysfunction due to corneal dystrophy, trauma or intraocular surgery.

The compound of the present invention and corneal endothelial cells and the like to be used for the implant for corneal endothelial keratoplasty of the present invention can be in any form similar to those of the above-mentioned therapeutic agent, corneal endothelial preparation and the like of the present invention.

In other aspect, the present invention provides a method of treating corneal endothelial dysfunction, comprising a step of providing a corneal endothelial preparation and/or an implant for corneal endothelial keratoplasty, each containing the compound of the present invention, and a step of transplanting the corneal endothelial preparation and/or the implant for corneal endothelial keratoplasty into a subject in need of corneal endothelial keratoplasty. In a preferable embodiment, the compound of the present invention can be compound (Ia) ((R)-(+)-N-(1H-pyrrolo[2,3-b]pyridin-4-yl)-4-(1-aminoethyl)benzamide or a pharmacologically acceptable salt thereof). The corneal endothelial preparation and implant for corneal endothelial keratoplasty to be used for the treatment method of the present invention can be in any form similar to those of the above-mentioned corneal endothelial preparation and implant for corneal endothelial keratoplasty. The treatment method of the present invention is useful for the treatment of corneal endothelial dysfunction, for example, bullous keratopathy, corneal edema, corneal leukoma and the like.

The administration (transplantation) subject of the corneal endothelial preparation of the present invention is, for example, a mammal (e.g., human, mouse, rat, hamster, rabbit, cat, dog, bovine, horse, sheep, monkey etc.), and human is preferable.

In the transplantation step, allogeneic transplantation is preferable, and a corneal endothelial preparation derived from corneal endothelial cells allogeneic with the animal to be the transplantation subject is preferably prepared. When the subject is human, a corneal endothelial preparation derived from a donor having the same blood type or HLA type is preferable, and autologous transplantation is more preferable.

In another aspect, the present invention provides an apoptosis suppressor containing the compound of the present invention. Here, the compound of the present invention can be preferably compound (Ia) ((R)-(+)-N-(1H-pyrrolo[2,3-b]pyridin-4-yl)-4-(1-aminoethyl)benzamide or a pharmacologically acceptable salt thereof). The compound of the present invention to be used for the apoptosis suppressor of the present invention can be in any form similar to those of the above-mentioned therapeutic agent of the present invention.

The apoptosis suppressor of the present invention has an effect to suppress development or progression of apoptosis, and is useful for the treatment or prophylaxis of a disease or pathology caused by hyper-abnormality of apoptosis, or a disease or pathology consequently showing such condition. Examples of the disease associated with hyper-abnormality of apoptosis include viral infections, endocrine diseases, hematological diseases, organ hypoplasia, organ graft rejection, graft-versus-host disease, immunodeficiency, neurodegenerative disease, ischemic cardiac diseases, radiation disorder, ultraviolet injury, poisoning diseases, malnutrition, inflammatory disease, ischemic neuropathy, vascular disease, respiratory diseases, articular syndrome and the like. Since the apoptosis suppressor of the present invention can particularly promote wound healing of corneal endothelial cells by suppression of cell apoptosis, it is useful for the treatment or prophylaxis of corneal endothelial dysfunction. Without particular limitation, the apoptosis suppressor of the present invention can contain an additive (stabilizer, solubilizer, suspending agent etc.) similar to those for the above-mentioned therapeutic agent. The content, dose, subject of administration and the like of the compound of the present invention as an active ingredient can also be similar to those for the above-mentioned therapeutic agent.

### EXAMPLES

The present invention is explained in more detail in the following by referring to Examples, which are not to be construed as limitative. The experimental animals were used according to the International Guiding Principles for Biomedical Research Involving Animals, as well as Act on welfare and management of animals, and standard relating to feeding, keeping and the like of experimental animals. This experiment was performed according to Guidelines of the Association for Research in Vision and Ophthalmology on the Use of Animals in Ophthalmic and Vision Research.

### Preparation Example

### Preparation Example of Test Substance

The composition of a test substance at each concentration is shown below.

Test substance

| compound (I) | 0.003, 0.01, 0.03, | |
|---|---|---|
| | 0.05 or 0.1 g | |
| | (content as dehydrochlorinated | |
| | form) | |
| sodium chloride | 0.85 | g |
| sodium dihydrogenphosphate dihydrate | 0.1 | g |
| benzalkonium chloride | 0.005 | g |

| | |
|---|---|
| sodium hydroxide | e.q. |
| purified water | e.q. |
| | total amount 100 ml (pH 7.0) |

Vehicle

| | | |
|---|---|---|
| sodium chloride | 0.85 | g |
| sodium dihydrogenphosphate dihydrate | 0.1 | g |
| benzalkonium chloride | 0.005 | g |

| | |
|---|---|
| sodium hydroxide | e.q. |
| purified water | e.q. |
| | total amount 100 ml (pH 7.0) |

### Example 1

### Effect of Compound (I) in Rabbit Corneal Endothelial Wound Model

### 1. Test Substance and Control Substance

As a test substance, (R)-(+)-N-(1H-pyrrolo[2,3-b]pyridin-4-yl)-4-(1-aminoethyl)benzamide monohydrochloride (compound (I)) was used. Compound (I) was produced according to the methods described in WO95/28387 and WO2002/083175.

In this Example, 0.95 mM (0.03 w/v%) compound (I) instillation and 0.32 mM (0.01 w/v%) compound (I) instillation obtained by diluting the above instillation with the above-mentioned vehicle were used.

As a positive control substances, Y-27632 dihydrochloride (Wako Pure Chemical Industries, Co., Ltd., Cat. #253-00513) and fasudil hydrochloride hydrate injection (Eril (registered trade mark) drip intravenous injection, Asahi Kasei Pharma) were purchased, and Y-27632 and Fasudil were each adjusted to 10 mM with phosphate-buffered saline (PBS, Invitrogen, Cat. #14190). In addition, PBS was used as a negative control substance.

### 2. Animal

Male Japanese white rabbits (body weight 2.5-3.0 kg, 21 rabbits) were purchased from Biotek Co., Ltd. and used. They are kept in individual cages at 23±3° C. temperature, at 55±10% humidity and 12 hours artificial light cycle (lighting on; 8:00 a.m., lighting off; 8:00 p.m.). One hundred grams of solid food (Labo R stock; Nosan Corp.) are provided daily to each animal. Water is supplied by automatic watering apparatus.

3. Excision of Nictitating Membrane of Animal Before starting the test, the nictitating membrane of both eyes of the animal was excised. To be specific, each animal was set in a positioner, and the ocular surface was topically anesthetized by instillation of a topical anesthesia (Benoxil instillation 0.4%, Santen Pharmaceutical Co., Ltd.). Then, the root position of the nictitating membrane was pressed with a clamp for 30 sec, and compressing mark was cut with scissors. After excision of the nictitating membrane, the antibiotic ointment (tarivid eye ointment, Santen Pharmaceutical Co., Ltd.) was instilled to prevent infection. After 4 days from the excision of the nictitating membrane, animals confirmed to have no abnormality in the external eye including the excised site were used.

### 4. Animal Grouping

The corneal thickness of the right eye of each animal was measured with an ultrasonic pachymeter (manufactured by DGH Technologies Inc., DGH-500), and the animals were divided into 4 groups such that the corneal thickness of each group was equal. The animals used for each group were as follows.
10 mM Y-27632 group=5 eyes
0.32 mM compound (I) group=5 eyes
0.95 mM compound (I) group=6 eyes
10 mM Fasudil group=5 eyes
PBS group=21 eyes (left eye)

### 5. Production of Corneal Endothelial Wound

The animals were sytemically anesthetized by intramuscular administration of Ketalar muscular injection (500 mg per 1 kg body weight, DAIICHI SANKYO COMPANY, LIMITED., 0.6 ml) and Celactal 2% Injection (Bayer, Ltd., 0.25 ml). Then, Benoxil instillation 0.4% (Santen Pharmaceutical Co., Ltd., one drop) was instilled and the eye was opened using a lid speculum.

A 7-mm diameter stainless dowel that had been cooled in liquid nitrogen was placed on the central cornea of both eyes of 21 animals for 15 sec. to produce an ice ball in the anterior chamber and dissociate corneal endothelial cells, whereby a corneal endothelial wound was produced.

### 6. Topical Instillation

For topical instillation, after wound creation, compound (I) instillation, Y-27632 instillation or Fasudil instillation was instilled to the right eye, and PBS was instilled to the left eye 6 times a day (4 times on the day of wound production) at 50 μL per instillation. Administration interval in one day was 2 hours.

### 7. Collection of Cornea and Measurement of Wound Area

After 46 hours from the wound creation, the rabbits were euthanized by injecting an overdose of 5% pentobarbital sodium solution (pentobarbital (Nacalai Tesque, Cat. #26427-14) dissolved in saline) in the marginal ear vein of the rabbits, and cornea tissue was excised. The cornea endothelial cells of the excised cornea were stained with 0.5% alizarin red S solution (Nacalai Tesque, Cat. #01303-52) and then examined under a microscope, and the stained images of the wound site were taken by an optical microscope (Olympus, BX51). The wound site was measured using image analysis software Image J (NIH, ver.1.41o), the outer circumference of the wound region stained with alizarin red was plotted by a manual operation, and the area surrounded by the plot was calculated as a wound area. The wound area was applied to statistical analysis (Ekuseru-Toukei 2008 for Windows, Social Survey Research Information Co., Ltd., ver. 1.10) according to the Dunnett's multiple comparisons test (both sides), and P values<0.05 were considered statistically significant.

### RESULTS AND DISCUSSION

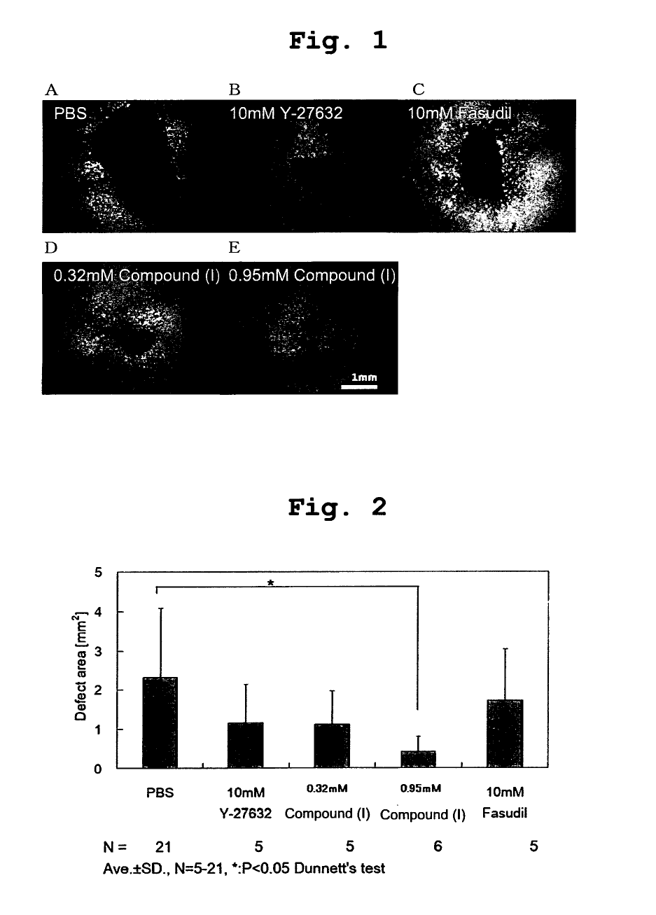

The alizarin stained images of the corneal endothelial wound sites after 46 hours from the wound creation are shown in FIG. 1, and the wound areas of the corneal endothelium are shown in FIG. 2. The area of the unrepaired wound site was 2.3 mm² by the PBS instillation group, and the smallest value of 0.4 mm² in the 0.95 mM compound (I) instillation group. As compared to the PBS instillation group, a statistically significant decrease in the wound area was observed. In addition, the wound area was as small as 1.1 mm² in the 0.32 mM compound (I) instillation group, as compared to the PBS instillation group, and was of the same level as 1.1 mm² of the 10 mM Y-27632 instillation group. On the other hand, the 10 mM

Fasudil instillation group showed a wider unrepaired wound area of 1.7 mm² than Y-27632 instillation group.

The above-mentioned results reveal that compound (I) reduces the wound area in the corneal endothelium at a concentration lower than that of Y-27632, suggesting the possibility of promoting wound healing.

### Example 2

### Effect of Administration of High Concentration of Compound (I) on Rabbit Corneal Endothelial Wound Model

The effect can be confirmed in rabbit corneal endothelial loss model by instillation of 0.05 w/v % (1.58 mM) compound (I) (topical instillation; 6 times a day, 2 days).

### Example 3

### Preparation of Rabbit Corneal Endothelial Cells

Male Japanese white rabbit eyeball tissues (collection target: about 2.5 kg of body weight) were purchased from Fukusaki Rabbit Warren and used. The 20 eyeballs were used. The cornea tissue was excised from the obtained rabbit eyeball tissues, and the Descemet's membrane was stripped together with intact corneal endothelial cells. The separated Descemet's membrane was incubated together with collagenase A (2.5 mg/mL, Roche, Cat. #1088793) under conditions of 37° C., 5% CO₂ for 2 hours. Thereafter, the cells were collected by centrifugation (1000 rpm (×70 g), 3 min.). The collected cells were diluted with a culture medium (DMEM (Invitrogen, Cat. #12320-032), 10% FBS and 2 ng/mL bFGF (Invitrogen, Cat. #13256-029), and 1% penicillin/streptomycin (Invitrogen, Cat. #15070-063)), seeded at a density of 2 eyes per well in a 6 well plate (Corning Incorporated, Cat. #3516) coated with FNC coating mix (Athena ES, Cat. #0407), and the cells were cultured until confluence at 37° C.

### Example 4

### Effect on the Morphology of Cultured Corneal Endothelial Cells

In this Example, compound (I), Y-27632 dihydrochloride (Wako Pure Chemical Industries, Co., Ltd., Cat. # 253-00513) and Fasudil (SIGMA-ALDRICH, Cat. #H139) were dissolved in DMSO (Nacalai Tesque, Cat. #13406-55), and further diluted with culture medium to give the test substances and control substance to be used.

The rabbit corneal endothelial cells prepared in Example 3 were washed twice with phosphate-buffered saline (PBS, Invitrogen, Cat. #14190), PBS (4 ml) was added, and the cells were incubated at 37° C. for 10 min. PBS was removed, 0.05% trypsin/EDTA (Invitrogen, Cat. #25300-054) was added, and the cells were incubated at 37° C. for about 5 min. A culture medium (10 ml, DMEM (Invitrogen, Cat. #12320-032), 10% FBS, 2 ng/mL bFGF (Invitrogen, Cat. #13256-029), and 1% penicillin/streptomycin (Invitrogen, Cat. #15070-063)) was added thereto, and the cells were collected in a tube, and centrifuged (1000 rpm (×70 g), 3 min.). The collected cells were diluted with a culture medium (about 3-4 ml).

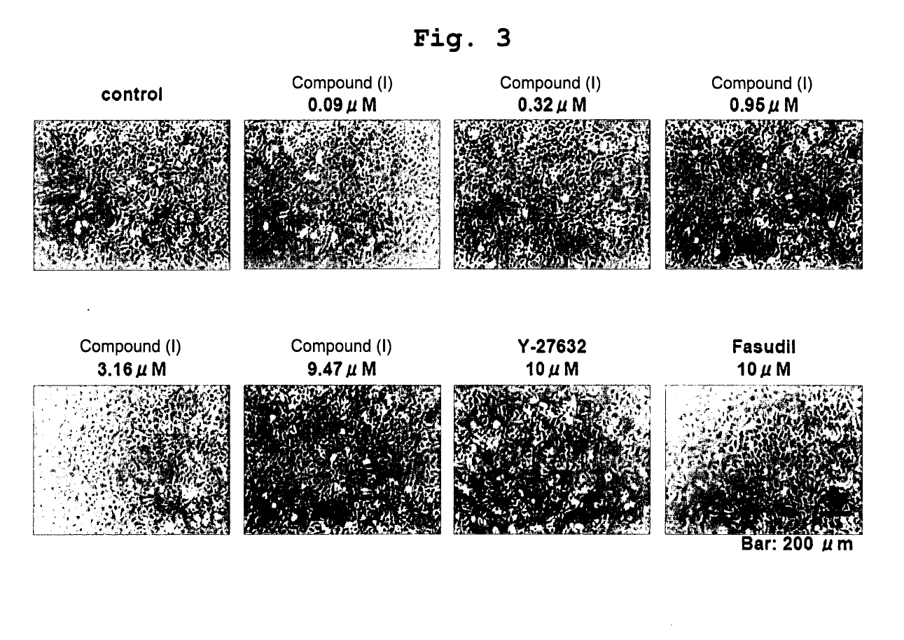

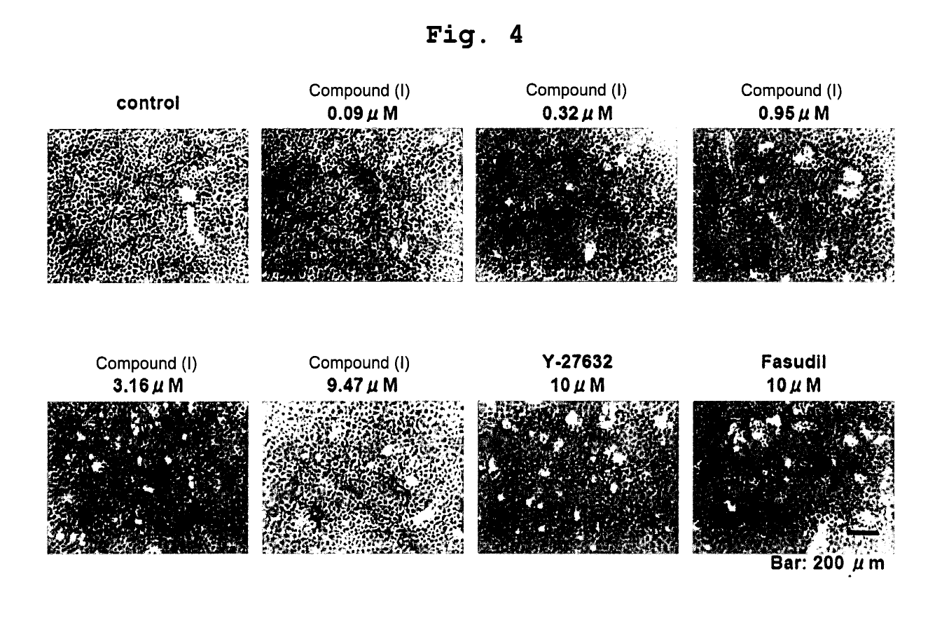

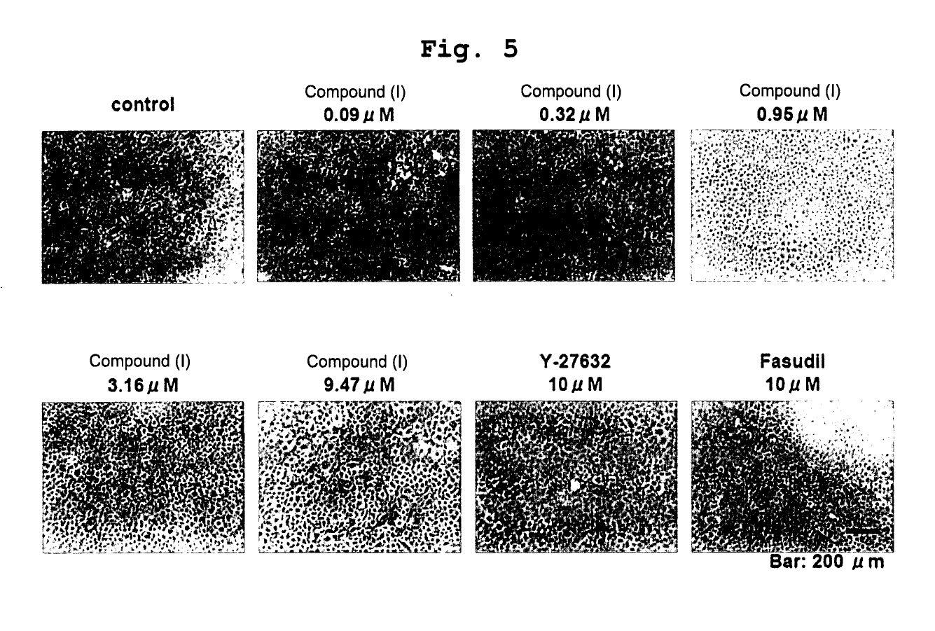

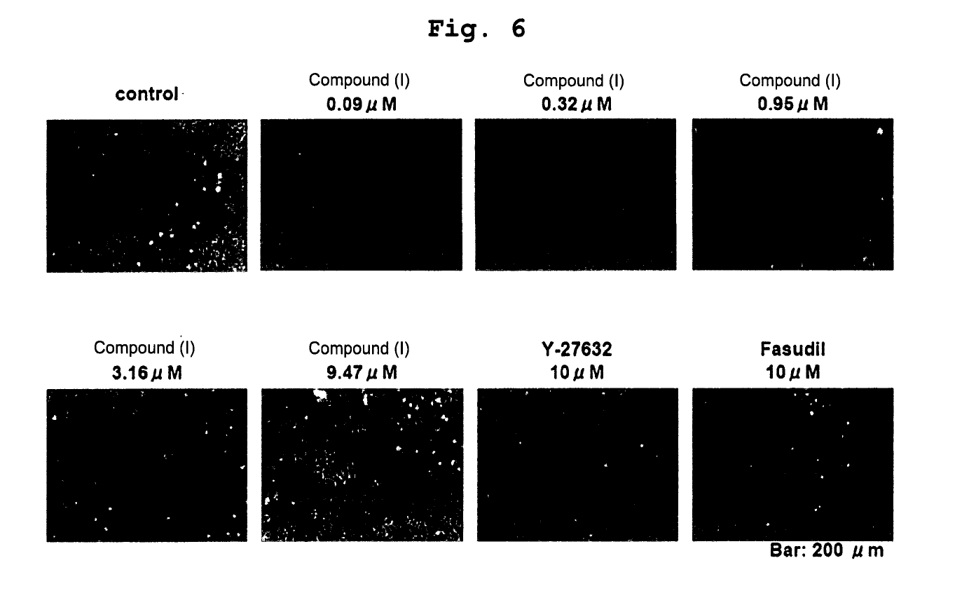

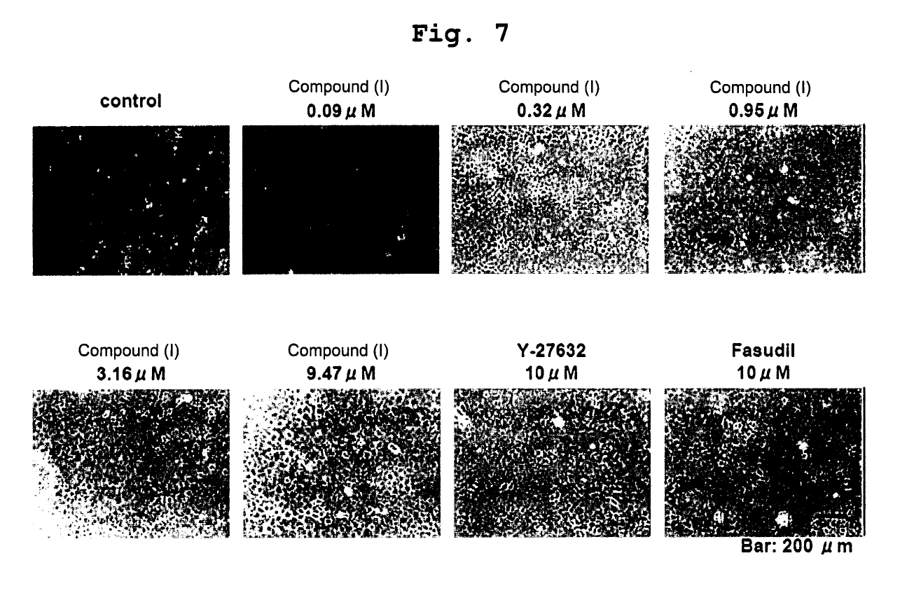

To the diluted rabbit corneal endothelial cells was added each medicament to final concentrations of 0.09, 0.32, 0.95, 3.16 and 9.47 μM compound (I), 10 μM Y-27632 and 10 μM Fasudil. These cells were seeded at a division ratio of 1:8 by 1 ml per well in a 24 well plate (Corning Incorporated, Cat. #3526). As a control, 0.04% DMSO/culture medium was added. After 1, 3, 5, 7, and 14 days from the addition, images were taken with a microscope. The results are shown in FIGS. 3 - 7.

By observation up to 14 days after the seeding, the cells had polymegethism and the formation of junction between cells was insufficient in the control group. In the 0.32, 0.95 and 3.16 μM compound (I) addition groups, the morphology of 5 corneal endothelial cells was maintained even after passaging, junction between cells was formed by 14 days after seeding, and a single cell layer was formed.

The results of 10 μM Y-27632 and Fasudil were similar to those of 0.32, 0.95 and 3.16 μM compound (I).

Compound (I) is considered to retain the morphology of corneal endothelial cells at a concentration lower than that of Y-27632 and Fasudil.

### Example 5

### Consideration in Wound Healing Model of Cultured Corneal Endothelial Cells

In this Example, rabbit corneal endothelial cells prepared in the same manner as in Example 4 were used.

In this Example, moreover, test substances and control substance prepared in the same manner as in Example 4 were used.

Collected rabbit corneal endothelial cells were diluted with a culture medium (about 4 ml, DMEM (Invitrogen, Cat. #12320-032), 10% FBS, 2 ng/mL bFGF (Invitrogen, Cat. #13256-029) and 1% penicillin/streptomycin (Invitrogen, Cat. #15070-063)), and seeded at a division ratio of 1:4 by 2 ml per well in a 6 well plate (Corning Incorporated, Cat. #3516). The cells were incubated to confluent under conditions of 37° C. and 5% CO₂. Wound was created in the confluent cells using 1000 μL chip (Molecular BioProducts, 2279). Then, each medicament was added to the culture medium to a final concentration of 0.09, 0.32, 0.95, 3.16 and 9.47 μM compound (I), 10 μM Y-27632 and 10 μM Fasudil. As a control, DMSO was added to a final concentration of 0.04%.

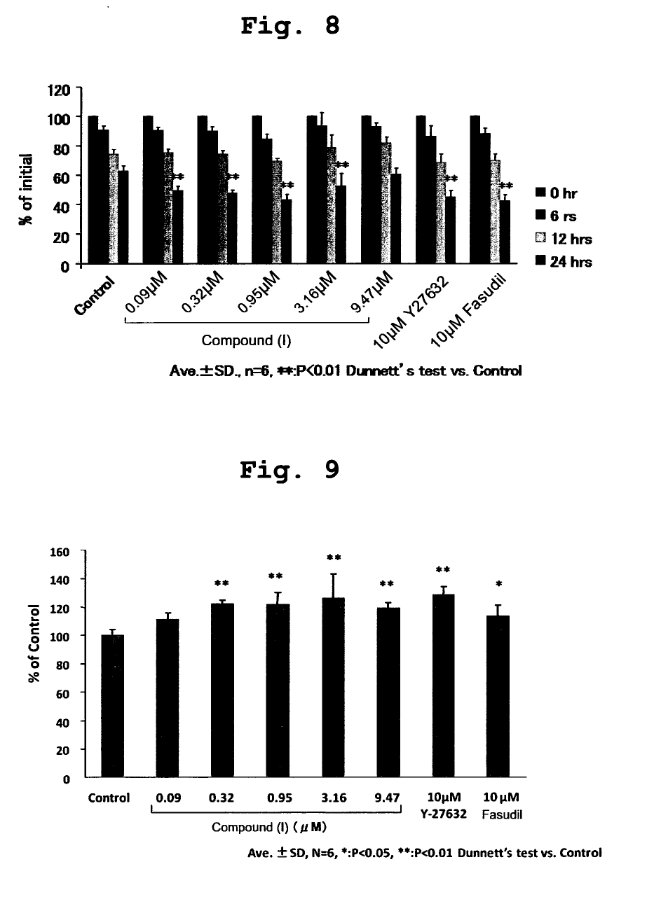

At 6 hours, 12 hours and 24 hours after addition of each medicament, the width of the wound was photographed over time, and the width of the wound was evaluated by image analysis. The results are shown in FIG. 8.

In the compound (I) addition group, wound width significantly decreased after 24 hours from the addition at a low concentration (0.09-3.16 μM), according to the Dunnett's test (FIG. 8). At 9.47 μM, no statistically significant difference from the control group was observed. According to the student's t-test, 0.95 μM compound (I) showed a wound healing effect from the early stages of the addition.

Compound (I) is considered to show a wound healing effect at a concentration (0.09-3.16 μM) lower than that of Y-27632 and Fasudil. From the above results, compound (I) also promoted wound healing in vitro corneal endothelium wound healing model.

### Example 6

### Effect on Adhesion Activity of Cultured Corneal Endothelial Cells to Culture Plate.

In this Example, rabbit corneal endothelial cells prepared in the same manner as in Example 4 were used.

In this Example, moreover, test substances and control substance prepared in the same manner as in Example 4 were used.

The collected rabbit corneal endothelial cells were diluted, and a culture medium (about 4 ml, DMEM (Invitrogen, Cat. #12320-032), 10% FBS, 2 ng/mL bFGF (Invitrogen, Cat. #13256-029) and 1% penicillin/streptomycin (Invitrogen, Cat. #15070-063)) was added to the cells. Then, each medicament was added to the culture medium to final concentrations of 0.09, 0.32, 0.95, 3.16 and 9.47 μM compound (I), 10 μM Y-27632 and 10 μM Fasudil. As a control, DMSO was added to a final concentration of 0.04%.

These cells were seeded in a 96 well plate (Corning Incorporated, Cat. #3595) at 1000 cells/well. After 3 hours from the seeding, suspended cells were removed, and adhered cells were counted by CellTiter-Glo (registered trade mark) Luminescent Cell Viability Assay (promega Cat. #G7572).

The results are shown in FIG. 9. All compound (I) addition groups increased the number of adherent cells in vitro. 0.32 μM compound (I) showed the number of adherent cells of almost the same level as 10 μM Y-27632. The number of adherent cells of compound (I) reached plateau at the concentration of not less than 0.32 μM.

### Summary of Examples 4-6

Compound (I) showed effects in cell adhesion, morphology and wound healing of corneal endothelial cells under the culture conditions, particularly at 0.32, 0.95 and 3.16 μM. Among these, the wound healing model showed a wound healing effect from early stages after the addition of 0.95 μM compound (I).

From these results, it is clear that compound (I) has effects of maintaining the morphology of corneal endothelial cells, promoting adhesion thereof, and treating a wound, at a lower concentration, as compared to Y-27632 and Fasudil.

### Example 7

### Production of Cultured Corneal Endothelial Cell Sheet for Transplantation

In this Example, rabbit corneal endothelial cells prepared in the same manner as in Example 4 were used.

In this Example, moreover, test substances and control substance prepared in the same manner as in Example 4 were used.

The rabbit corneal endothelial cells were seeded on Vitrigel™ (Asahi Glass) at a division ratio of 1:1, and a cultured corneal endothelial cell sheet for transplantation was produced. When producing the corneal endothelial cell sheet, 0.95 μM compound (I), 10 μM Y-27632 or 0.04% DMSO was added. The obtained corneal endothelial cell sheet was subjected to fluorescence immunostaining with ZO-1 and Na⁺/K⁺ ATPase, which are functional proteins of corneal endothelial cells, and expression was confirmed.

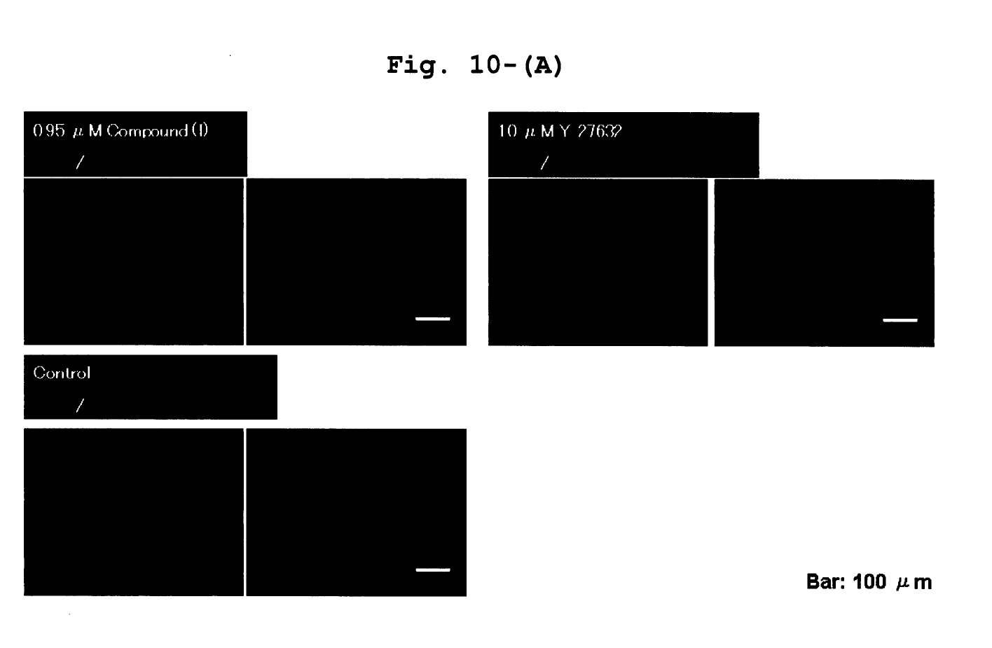

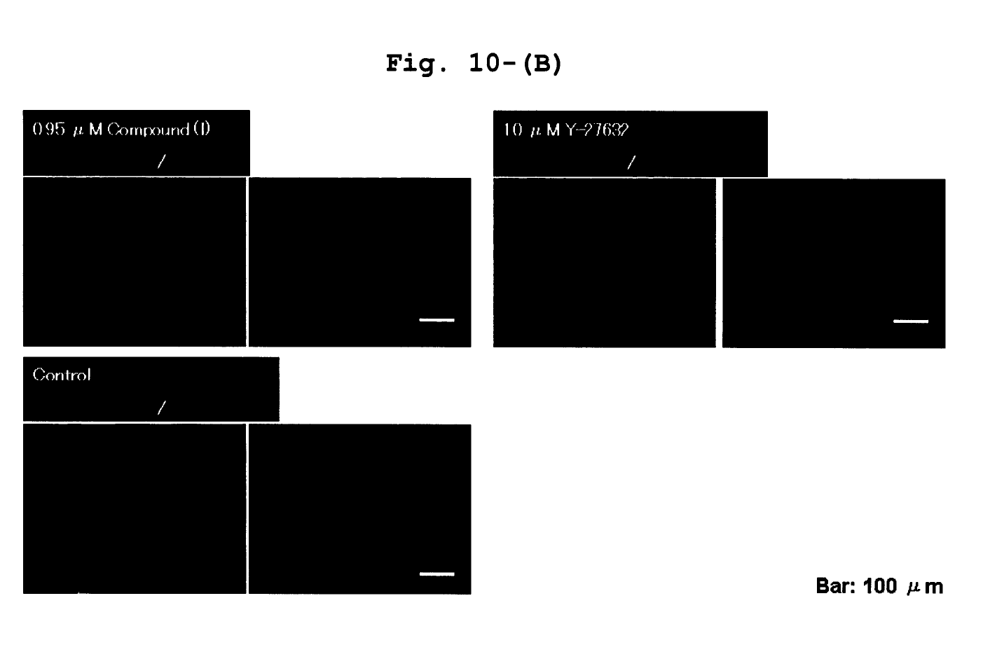

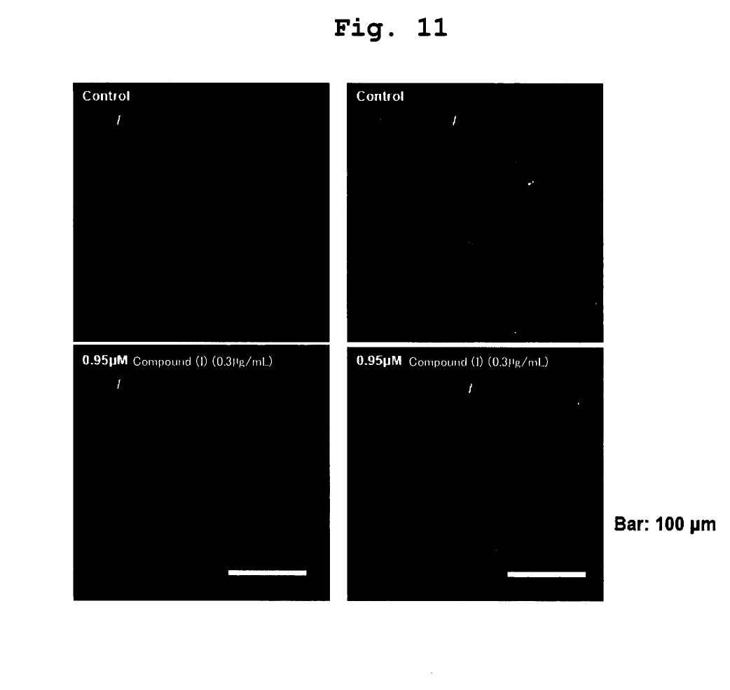

The corneal endothelial cell sheet was fixed with 95% ethanol (−30° C.) for 10 min, washed with PBS, and treated with 0.5% Triton X-100/PBS for 5 min. Then, it was treated with 1% BSA/PBS for 1 hour, and treated overnight with anti-ZO-1 antibody (Invitrogen, Cat. #339100) or anti-Na⁺/K⁺ ATPase antibody (Millipore, Cat. #C464.6). After washing with PBS, it was treated with Alexa-488 labeling secondary antibody for 1 hour. After washing with PBS, mounting medium (Vectashield (registered trade mark)) containing DAPI was added dropwise to the sheet, and the sheet was sealed with cover glass. The image was taken with a fluorescence microscope to confirm expression of ZO-1 and Na⁺/K⁺ ATPase. The immunostaining was performed both 48 hours and 14 days after the seeding (FIG. 10 and FIG. 11).

### Formulation Example 1

### Culture Medium for Preparation of Corneal Endothelium Sheet Containing Compound (I)

In this Example, a culture medium shown below was prepared according to a conventional method and used.

| | | |
|---|---|---|
| compound (I) | 0.5 | mg |
| FBS | 10 | mL |
| penicillin/streptomycin solution | 1 | mL |
| FGF basic | 200 | ng |

| | |
|---|---|
| DMEM | e.q. |
| | total amount 100 mL |

Used were FBS manufactured by Invitrogen, penicillin/streptomycin solution manufactured by Invitrogen (containing 5000 U/mL penicillin, 5000 μg/mL streptomycin), FGF basic manufactured by Invitrogen, compound (I) manufactured by Mitsubishi Tanabe Pharma Corporation, and DMEM manufactured by Invitrogen.

The results at 48 hours after the seeding showed that ZO-1, which is an index of barrier function of the corneal endothelium, was expressed between cells. The expression of ZO-1 was found between cells uniformly in the groups added with compound (I) and Y-27632. However, in the group added with DMSO (control group), the expression could only be confirmed in a partial cell lump (FIG. 10-(A)). In addition, Na⁺/K⁺ ATPase, which is an index of corneal endothelium pumping function, was located between cells. The expression of Na⁺/K⁺ ATPase was also found between cells in all in the groups added with compound (I) and Y-27632. However, in the control group, the expression could only be confirmed in a partial cell lump (FIG. 10-(B)).

The above results reveal that adhesion between cells was insufficient in the control group at 48 hours after the seeding. On the other hand, adhesion between cells was formed in the Y-27632 treatment group and the expression of ZO-1 and Na⁺/K⁺ ATPase, which are functional proteins, was confirmed at the adhesion site. However, in the Y-27632 treatment group, the sheet was partially uncovered with cells, and was insufficient as a cultured corneal endothelial cell sheet for transplantation.

In contrast, in the compound (I) treatment group, expression of ZO-1 and Na⁺/K⁺ ATPase was confirmed in the adhesion site formed between cells, and cells were adhered to the whole surface of the sheet. Thus, the sheet was considered to be sufficiently usable as a cultured corneal endothelial cell sheet for transplantation. Hence, it was found that, by adding compound (I) to a culture medium, a cultured corneal endothelial cell sheet for transplantation can be produced in 48 hours from the addition. The above demonstrates that, using compound (I), a cultured corneal endothelial cell sheet suitable for transplantation can be produced at an early stage.

### Example 8

### Effect of Rabbit Corneal Endothelial Cell Injection Therapy Using Compound (I) on Rabbit Bullous Keratopathy Model

### (1) Production of Babbit Bullous Keratopathy Model

Male Japanese white rabbits (Biotek Co., Ltd., 8 rabbits) were subjected to the following nictitating membrane excision. Each animal was set in a positioner, and the ocular surface was topically anesthetized by instillation of a topical anesthesia (Benoxil instillation 0.4%, Santen Pharmaceutical Co., Ltd.). Then, the root position of the nictitating membrane was pressed with a clamp for 30 sec, and compressing mark was cut with scissors. After excision of the nictitating membrane, the antibiotic ointment (tarivid eye ointment, Santen Pharmaceutical Co., Ltd.) was instilled to prevent infection.

Three days after nictitating membrane excision, phacoemulsification and aspiration surgery (PEA) was performed on the left eye. Under systemic anesthesia, 3 mm incision was formed in the corneoscleral limbus, crystalline lens was excised by a cataract surgery instrument (NIDEK Co., Ltd.), and the incision was sutured with a nylon thread (Mani Inc.). After phacoemulsification and aspiration surgery (PEA), the antibiotic ointment (tarivid eye ointment, Santen Pharmaceutical Co., Ltd.) was instilled to prevent infection. After 5 days from the PEA, the animals were systemically anesthetized by intramuscular administration of Ketalar muscular injection (500 mg per 1 kg body weight, DAIICHI SANKYO COMPANY, LIMITED., 0.6 ml) and Celactal 2% Injection (Bayer, Ltd., 0.25 ml). Then, Benoxil instillation 0.4% (Santen Pharmaceutical Co., Ltd., one drop) was instilled and the eye was opened using a lid speculum. Thereafter, 1 mm incision was formed in the corneoscleral limbus, and corneal endothelial cells were scraped with a silicon surgical instrument to mechanically detach the cells. The detachment area was confirmed by trypan blue staining.

### (2) Injection of Corneal Endothelial Cells to Rabbit Bullous Keratopathy Model

The corneal endothelial cells were mechanically scraped, the cultured corneal endothelial cells were collected with 0.05% trypsin-EDTA (Invitrogen, Cat. #25300-054) from the culture flask to give a cell suspension. Using Dulbecco's modified Eagle's medium (DMEM) (Invitrogen, 12320-032), the cultured rabbit corneal endothelial cells were suspended in 3 groups of 10 μM compound (I)/DMEM, 100 μM Y-27632/DMEM and DMEM, each at 1.0×10⁶ cells/ml. The cell suspension of each group (200 μl (2.0×10⁵ cells per one eye)) was injected with a 22 gauge needle to the anterior chamber from the corneoscleral limbus of the prepared rabbit bullous keratopathy model, and the rabbit was fixed looking downward such that the corneal endothelium face was on the upper side and the corneal epithelial face was on the lower side for 3 hours. The fixing with looking downward was performed with appropriate addition of anesthetics, paying sufficient attention to animal protection.

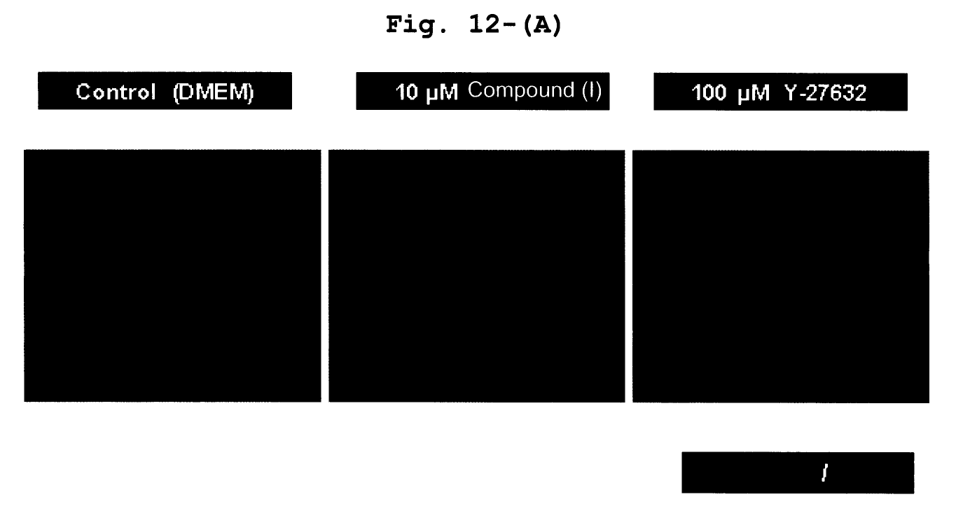

After 14 days from the cell injection, the treated eye was isolated, and a corneoscleral tissue was excised from the isolated eyeball. The obtained corneoscleral tissue was fixed with 4% para-formaldehyde/PBS for 10 min, and blocked overnight with PBS (Invitrogen, Cat. #14190-144) containing 1% BSA (SIGMA, Cat. #A7906-50G). Thereafter, the corneoscleral tissue was divided into two, and treated with Alexa-488 labeling phalloidin staining actin (Invitrogen, Cat. #Al2379), or anti-Na⁺/K⁺ ATPase antibody, which is a corneal endothelium marker (UP State, Cat. #05-369), for 2 hours. The anti-Na⁺/K⁺ ATPase antibody treatment group was further treated with Alexa-488 labeled secondary antibody (Invitrogen, Cat. #A-21202) for 1 hour. Thereafter, the cells were immersed in Vectashield (registered trade mark)-DAPI (Vector Laboratories, Cat. #H-1200) solution, and mounted using cover glass. The specimen was observed under a confocal laser microscope. The stained images are shown in FIG. 12.

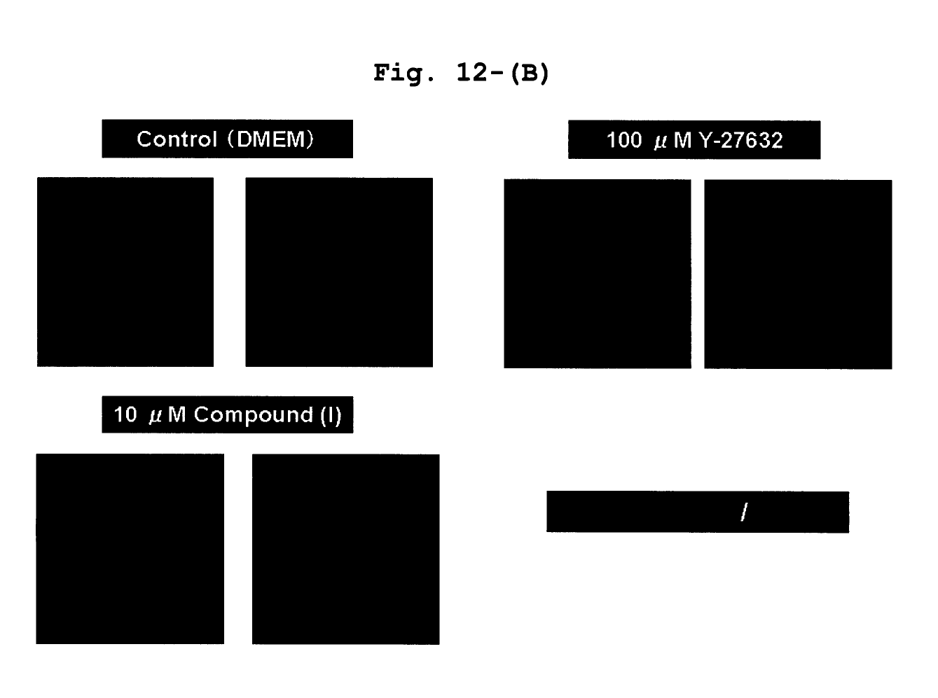

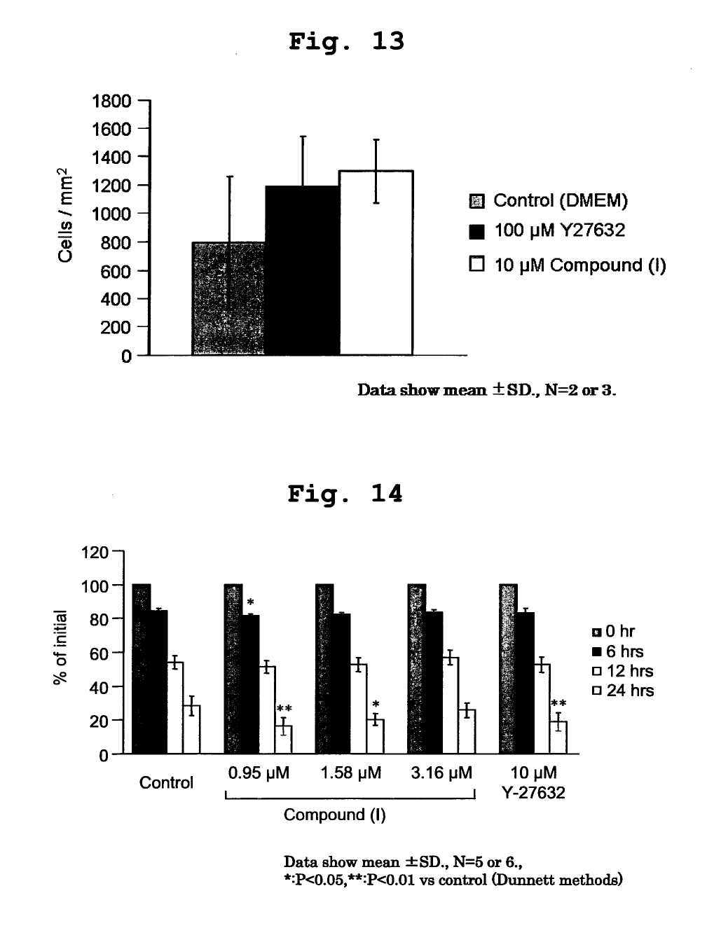

Taking the number of nuclei stained with DAPI as the corneal endothelial cell count, the DAPI stained images were analyzed by Image-Pro plus (Media Cybernetics, Inc.), and corneal endothelial cells were counted. The results are shown in FIG. 13.

From the results of Phalloidin staining, it was found that the injected cultured corneal endothelial cells developed fibrosis in the control group treated with DMEM alone; however, the 10 μM compound (I) treatment group showed suppression of fibrosis of cells in the same manner as in the 100 μM Y-27632 treatment group (FIG. 12-(A)). In addition, as compared to the control group, expression of Na⁺/K⁺ ATPase was found in the 10 μM compound (I) treatment group and 100 μM Y-27632 treatment group (FIG. 12-(B)). The corneal endothelial cells were counted. As a result, the 10 μM compound (I) treatment group and 100 μM Y-27632 treatment group tended to show higher cell counts than the control group, and the cell count of the 10 μM compound (I) treatment group was higher than that of the 100 μM Y-27632 treatment group (FIG. 13).

From the above results, 10 μM compound (I) is considered to show the highest culture effect of corneal endothelial cells, and is most suitable for the corneal endothelial cell injecting therapy.

### Example 9

### Wound Healing Effect of Compound (I) on Rabbit Corneal Endothelial Cells in Vitro

In this Example, test substances and control substance prepared in the same manner as in Example 4 were used.

### (1) Preparation of Rabbit Corneal Endothelial Cells

Corneoscleral tissue was collected from 10 rabbit eyeballs purchased from Funakoshi Corporation. The corneoscleral tissue was immersed in DMEM (Invitrogen, Cat. #12320-032) containing 1% penicillin/streptomycin (Invitrogen, Cat. #15140-122), and incubated at 37° C. for 1 hour. The Descemet's membrane was stripped together with intact corneal endothelial cells, immersed in a culture medium (DMEM, 10% FBS, 2 ng/mL bFGF (Invitrogen, Cat. #13256-029), 1% penicillin/streptomycin) containing 2 mg/mL Collagenase A (Roche, Cat. #1088793), and incubated at 37° C. for 2 hours. The cells were collected by centrifugation, washed with the culture medium, and seeded in a T25 flask (Corning Incorporated, Cat. #430639) coated with FNC coating mix (Athena ES, Cat. #0407). The flask were left standing in a 5% CO₂ incubator at 37° C., the medium was exchanged every 2-3 days, and the cells were cultured to confluent. The cells that reached confluent were collected, seeded in two 6 well plates (Falcon, Cat. #3046) coated with FNC coating mix, and cultured to confluent.

### (2) Production of In Vitro Wound Healing Model

The prepared rabbit corneal endothelial cells were collected, seeded in a 6 well plate at a division ratio of 1:4, and cultured to confluent in the same culture medium as in the above-mentioned (1). Linear wound was created in the confluent cells using 1000 μL chip (6 wounds per well).

### (3) Addition of Medicament and Evaluation of Wound Healing Effect

After creating the linear wounds, the culture medium was exchanged, and 0.95 μM, 1.58 μM and 3.16 μM compound (I), 10 μM Y-27632 and 0.04% DMSO were added. Compound (I) and Y-27632 were dissolved in DMSO in advance, where the DMSO concentration was adjusted to 0.04% for both.

The width of the wound at 0, 6, 12 and 24 hours after the addition was photographed over time. The wound width was measured by Image-Pro plus (Media Cybernetics, Inc.). The ratio of wound width at each hour was calculated by taking that at 0 hour after the addition of medicament as 100%, and the time-course changes of the ratio of wound width were evaluated. The ratio of wound width was applied to statistical analysis according to the Dunnett's test at each time point. The results are shown in FIG. 14.

As a result, compound (I) at a low concentration (0.95 μM) showed a statistically significant difference from the control after 6 hours from the addition of the medicament, suggesting a high wound healing effect. In addition, the compound (I) showed a statistically significant difference even 24 hours later, and at the same time Y-27632 also showed a difference from the control for the first time.

Therefore, compound (I) is considered to show a wound healing promoting effect at a concentration (0.95-1.58 μM) lower than that of Y-27632. While the 1.58 μM compound (I) also showed a significant effect, the effect was weaker than that of the 0.95 μM compound (I). Hence, the concentration of compound (I) showing the highest effect in the in vitro wound healing model is considered to be about 0.95 μM.

### Example 10

### Cell Death Suppressive Effect of Compound (I) Added to Corneal Storage Solution

In this Example, test substances and control substances prepared in the same manner as in Example 4 were used.

Both right and left eyeballs of 5 male Japanese white rabbits (Biotek Co., Ltd.) were removed, and corneoscleral tissues were prepared. One corneoscleral tissue was placed in a storage solution Optisol-GS (registered trade mark) (Bausch & Lomb, Inc) (control), and the other corneoscleral tissue was placed in Optisol-GS containing 0.95 μM compound (I). Similarly, corneoscleral tissues were prepared from another 5 male Japanese white rabbits, one corneoscleral tissue was placed in Optisol-GS (control), and the other corneoscleral tissue was placed in Optisol-GS containing 10 μM Y-27632.

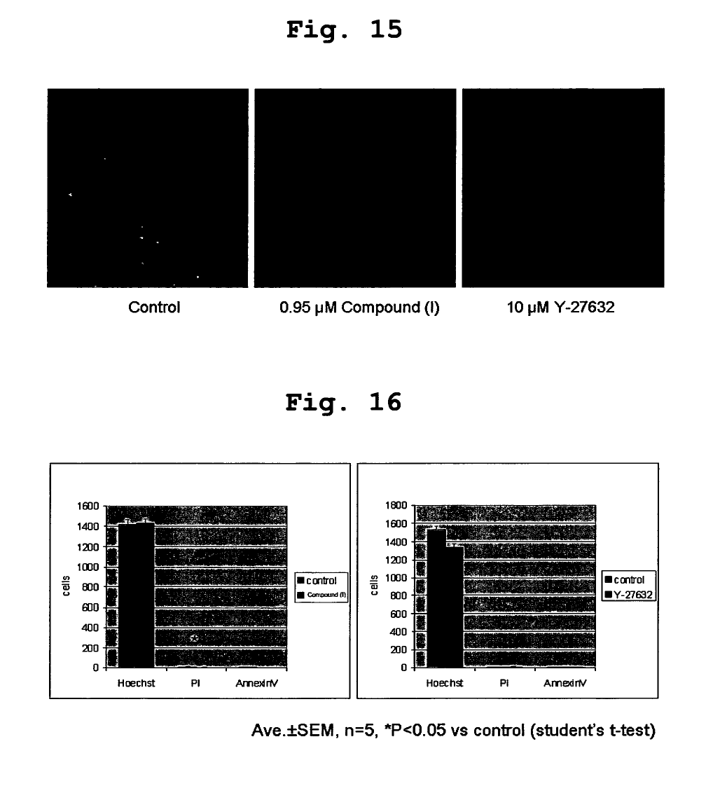

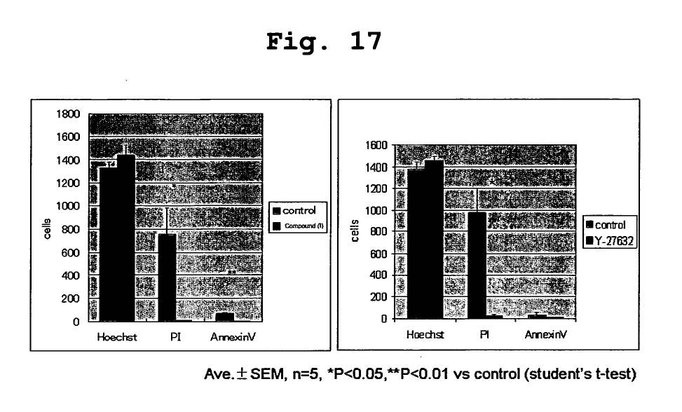

The samples containing each corneoscleral tissue were preserved at 4° C. Two or three weeks later, the corneoscleral tissues were stained with Hoechst (Hoechst 33342, Sigma, Cat. #B2261), PI (propidium iodide, Sigma, Cat. #P4170) and Annexin V (Annexin V-FITC, MBL, Cat. #4700-100), and the cells therein were indentified as living cells, dead cells and apoptotic cells. The stained images of the corneoscleral tissues in the samples 3 weeks later are shown in FIG. 15. In addition, the various cells in the stained cornea were counted using ImageJ (ver.1.44i, NIH, http://imagej.nih.gov/ij), and the mean value and the standard deviation of 5 visual fields were determined. The various cell counts were statistically analyzed by the student's t-test. The results 2 weeks later are shown in FIG. 16 and those 3 weeks later are shown in FIG. 17.

As a result, the number of dead cells in the storage solution containing compound (I) decreased significantly 2 weeks later as compared to the control. The results 3 weeks later reveal that the numbers of both the dead cells and apoptotic cells decreased significantly as compared to the control when the storage solution containing compound (I) was used. In contrast, when the storage solution containing Y-27632 was used, only the number of dead cells decreased significantly.

The above results indicate that, by adding compound (I) to a storage solution, the cell death and apoptosis can be suppressed more significantly than by conventional storage solutions. Moreover, it was demonstrated that compound (I) shows the effect at a concentration lower than that of Y-27632.

This application is based on patent application No. 2009-299180 (filing date: December 29, 2009) filed in Japan and international application No. PCT/JP2010/071424 (filing date: Nov. 24, 2010), the contents of which are incorporated in full herein by this reference.

1-8. (canceled)

9. A method of treating corneal endothelial dysfunction, comprising a step of providing a corneal endothelial preparation and/or an implant for corneal endothelial keratoplasty, each comprising (R)-(+)-N-(1H-pyrrolo[2,3-b]pyridin-4-yl)-4-(1-aminoethyl)benzamide or a pharmacologically acceptable salt thereof, and a step of transplanting the corneal endothelial preparation and/or the implant for corneal endothelial keratoplasty to a subject in need of corneal endothelial keratoplasty.

10. The method according to claim 9, wherein the corneal endothelial cells are derived from human.

11. A method of treating corneal endothelial dysfunction, comprising a step of administering an effective amount of (R)-(+)-N-(1H-pyrrolo[2,3-b]pyridin-4-yl)-4-(1-aminoethyl)benzamide or a pharmacologically acceptable salt thereof to a subject in need of corneal endothelial wound healing.

12. The method according to claim 11, wherein the administration step is topical instillation.

13-19. (canceled)
